(19) United States
(12) Patent Application Publication
(10) Pub. No.: US 2024/0319174 A1
(43) Pub. Date: Sep. 26, 2024

(54) ORGANOID CO-CULTURES AND METHODS OF USE THEREOF

(71) Applicants: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US)

(72) Inventor: Karuna Ganesh, New York, NY (US)

(73) Assignees: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US)

(21) Appl. No.: 18/732,216

(22) Filed: Jun. 3, 2024

Related U.S. Application Data
(63) Continuation of application No. PCT/US2022/051541, filed on Dec. 1, 2022.
(60) Provisional application No. 63/284,700, filed on Dec. 1, 2021.

(51) Int. Cl. G01N 33/50; C12N 1/20; C12N 5/0783; C12N 5/09; C12Q 1/02

(52) U.S. Cl. CPC G01N 33/5088; C12N 1/20; C12N 5/0636; C12N 5/0693; C12Q 1/025; C12N 2501/11; C12N 2501/415; C12N 2501/998; C12N 2503/02

(57) ABSTRACT

The present disclosure provides organoid co-cultures and methods of using such co-cultures. In particular, the present disclosure provides organoid-immune cell and organoid-bacterial cell co-cultures. The present disclosure further provides methods for testing therapeutic agents using the disclosed organoid co-cultures.

### CROSS-REFERENCE TO RELATED APPLICATIONS

This application is a continuation of International Application No. PCT/US2022/051541, filed Dec. 1, 2022, which claims priority to U.S. Provisional Application No. 63/284,700, filed Dec. 1, 2021, the contents of both of which are incorporated herein by reference in their entireties.

### GRANT INFORMATION

This invention was made with government support under grant number K08CA230213 awarded by the National Institutes of Health (NIH). The government has certain rights in the invention.

### BACKGROUND

Despite recent advances in cancer therapeutics such as the development of immunotherapy and chimeric antigen receptor (CAR) T cell therapy many challenges limit the therapeutic efficacy of such therapeutics. For example, such therapies can be limited by life-threatening toxicities, minimal anti-tumor activity and limited tumor infiltration. In addition, the interactions between T cells and the tumor microenvironment can affect the effectiveness of anti-tumor immunity. Therefore, there is a need in the art for model systems that can recapitulate the tumor microenvironment for identifying effective cancer therapeutics.

### SUMMARY

The present disclosure provides organoid co-cultures and methods for generating and using such organoid co-cultures. In particular, the present disclosure provides organoid-immune cell co-cultures and organoid-bacterial cell co-cultures.

The present disclosure provides methods for testing a therapeutic agent. In certain embodiments, the method includes (a) contacting an organoid co-culture with a candidate therapeutic agent, (b) detecting the presence of a change in the organoid co-culture that is indicative of the effectiveness of the therapeutic agent and (c) identifying the candidate therapeutic agent as likely to have a therapeutic effect if the change is detected.

In certain embodiments, the change is a change in cell viability, cell proliferation, organoid size, morphology, mutational status, epigenetic state, RNA expression levels and/or protein expression including secreted proteins such as cytokines or chemokines. In certain embodiments, the change is a change in cell viability, e.g., a change in the viability of the cells of the organoid.

In certain embodiments, the organoid is generated from cancer cells of a subject. In certain embodiments, the cancer cells are colorectal or esophagogastric cancer cells. In certain embodiments, the organoid co-culture comprises an immune cell or a bacterial cell. In certain embodiments, the organoid co-culture comprises an immune cell. In certain embodiments, the organoid co-culture comprises a bacterial cell. In certain embodiments, the immune cell is a T cell. In certain embodiments, the immune cell is a CAR T cell. In certain embodiments, the T cells are derived from the same subject that has the cancer. In certain embodiments, the bacterial cell is obtained from the same subject. In certain embodiments, the organoid comprises one or cancer cells that express high levels of LICAM. In certain embodiments, the CAR T cell is a LICAM CAR T cell. In certain embodiments, the ratio of the total number cells of the organoid to the total number of immune cells is from about 1:20 to about 20:1, e.g., about 1:1 to about 20:1. In certain embodiments, the ratio of the total number cells of the organoid to the total number of immune cells is about 1:10. In certain embodiments, the ratio of the total number cells of the organoid to the total number of immune cells is about 1:5. In certain embodiments, the ratio of the total number cells of the organoid to the total number of immune cells is about 1:2.

The present disclosure further provides methods for testing the efficacy of a candidate modified immune cell that include (a) contacting an organoid with the candidate modified immune cell to generate an organoid-immune cell co-culture, (b) detecting the presence of a change in the organoid-immune cell co-culture that is indicative of the effectiveness of the candidate modified immune cell and (c) identifying the candidate modified immune cell as likely to have a therapeutic effect if the change is detected. In certain embodiments, the modified immune cell is an immune cell genetically engineered to express an antigen-binding receptor. In certain embodiments, the antigen-binding receptor is a chimeric antigen receptor (CAR). In certain embodiments, the change is a change in cell viability, cell proliferation, organoid size, morphology, mutational status, epigenetic state, RNA expression levels and/or protein expression. In certain embodiments, the change is a change in cell viability, e.g., a decrease in cell viability. In certain embodiments, the organoid is generated from cancer cells of a subject. In certain embodiments, the modified immune cells are derived from the subject. In certain embodiments, the organoid comprises one or more colorectal or esophagogastric cancer cells. In certain embodiments, the organoid comprises one or cancer cells that express high levels of LICAM. In certain embodiments, the modified immune cell is a CAR T cell. In certain embodiments, the CAR T cell is a LICAM CAR T cell.

The present disclosure provides methods for analyzing a bacterial species. In certain embodiments, the method includes (a) culturing an organoid with a candidate bacterial species to generate an organoid-bacterial cell co-culture, (b) detecting the presence of a change in the organoid-bacterial cell co-culture that is indicative of the oncogenic potential of the bacterial species and (c) identifying the bacterial species as likely to have an oncogenic effect if the change is detected. In certain embodiments, the change is a change in cell viability, cell proliferation, organoid size, morphology, mutational status, RNA expression levels and/or protein expression. In certain embodiments, the change is a change in cell viability. In certain embodiments, the change is a change in mutational status. In certain embodiments, the organoid-bacterial cell co-culture further comprises an immune cell, e.g., a T cell, e.g., a CAR T cell. In certain embodiments, the organoid is generated from normal cells of a subject.

In certain embodiments, the method for analyzing a bacterial species includes (a) providing an organoid-bacterial cell co-culture comprising cancer cells and one or more candidate bacteria, (b) contacting the organoid-bacterial cell co-culture with a therapeutic agent, (c) detecting the presence of a change in the organoid-bacterial cell co-culture that is indicative of the potential of the bacterial species to increase the effectiveness of the therapeutic agent and (d) identifying the bacterial species as likely to increase the effectiveness of the therapeutic agent if the change is detected. In certain embodiments, the organoid-bacterial cell co-culture further comprises an immune cell. In certain embodiments, the therapeutic agent is an immune cell. In certain embodiments, the immune cell is a T cell, e.g., a CAR T cell. In certain embodiments, the change is a change in cell viability, cell proliferation, organoid size, morphology, mutational status, epigenetic state, RNA expression levels and/or protein expression. In certain embodiments, the change is a change is a change in cell viability. In certain embodiments, the change is a decrease in cell viability of the cells of the organoid. In certain embodiments, the organoid is generated from cancer cells of a subject.

In certain embodiments, the organoid co-culture, the organoid-immune cell co-culture and/or the organoid-bacterial cell co-culture is cultured in a media comprising: (a) from about 1 to about 500 ng/ml of Wnt3a, (b) from about 1 to about 500 ng/ml of Noggin, (c) from about 1 to about 500 ng/ml of EGF, (d) from about 0.01 to about 1,000 ng/ml of R-spondin-1 or (c) a combination of any one of (a)-(d). In certain embodiments, the organoid co-culture is cultured in a media comprising about 100 ng/ml Wnt-3a, about 1,000 ng/ml R-spondin-1, about 50 ng/ml Noggin and about 50 ng/ml EGF.

In certain embodiments, the organoid co-culture, the organoid-immune cell co-culture and/or the organoid-bacterial cell co-culture is cultured in a media comprising an antibiotic at a concentration from about 0.005 μg/μl to about 5 μg/μl. In certain embodiments, the concentration of the antibiotic is from about 0.001 μg/μl to about 1.0 μg/μl. In certain embodiments, the antibiotic is gentamycin.

The present disclosure further provides an organoid co-culture comprising one or more organoid cells and one or more immune cells or bacterial cells, wherein the ratio of organoid cells to immune or bacterial cells is from about 1:20 to about 20:1, e.g., about 1:1 to about 20:1. In certain embodiments, the ratio of organoid cells to immune or bacterial cells is about 1:2. In certain embodiments, the ratio of organoid cells to immune or bacterial cells is about 1:5. In certain embodiments, the ratio of organoid cells to immune or bacterial cells is about 1:10.

The present disclosure further provides kits for performing methods of the present disclosure.

### BRIEF DESCRIPTION OF THE FIGURES

FIG. 1 provides data showing the targeting of LICAM-positive dissociated or intact organoids with LICAM CAR T cells.

FIG. 2 provides data showing the targeting of LICAM-positive dissociated or intact organoids with LICAM CAR T cells.

FIG. 3 provides an exemplary listing of genes to analyze in the LICAM-positive organoids.

FIGS. 4A-4F provides the expression level of B2M (FIG. 4A), HMGB1 (FIG. 4B), HSPA8 (FIG. 4C), HSPA5 (FIG. 4D), PDIA3 (FIG. 4E) and NFYC (FIG. 4F) in the organoid-CAR T cell co-cultures.

FIG. 5A provides an experimental method for generating organoids co-cultures with immune cells according to the present disclosure.

FIG. 5B provides the cytotoxicity of the CAR T cells after 36 hours.

FIG. 5C provides the expression level of LICAM in the organoids.

FIGS. 5D-5E provides the percent viability of the organoids in the presence of the CAR T cells.

FIG. 5F provides the cell count in the organoid-CAR T cell co-cultures.

FIG. 5G provides the population of cells that are PD-1-positive or TIM3+ in the organoid-CAR T cell co-cultures.

FIG. 5H provides the levels of cytokines in the organoid-CAR T cell co-cultures.

FIGS. 6A-6B show that co-culturing LICAM CAR T cells with organoids resulted in cytotoxicity of the organoids compared to control.

FIGS. 7A-7B provide images of the LICAM organoid-LICAM CAR T cell co-culture after 72 hours.

FIG. 8 provides an exemplary schematic of methods for isolating TILs and PBMCs according to the present disclosure.

FIG. 9 provides a schematic displaying the different populations of cells within the isolated TILs and PBMCs.

FIG. 10 provides the expression levels of TIM23, PD-1 and LAG3 in the isolated TILs and PBMCs.

FIG. 11 provides the cell number and viability after thawing of the isolated TILs and PBMCs.

FIG. 12A provides an exemplary protocol for generating patient-specific organoid-immune cell co-cultures according to the present disclosure.

FIGS. 12B-12D provide that an anti-PD-1 antibody enhances the T cell-mediated killing of organoids.

FIG. 13A provides an exemplary protocol for generating patient-specific organoid-immune cell co-cultures for use in testing therapeutic agents and combinations.

FIG. 13B provides the results of the combination of trastuzumab and an anti-PD-1 antibody on organoid viability.

FIG. 13C provides the results of the combination of trastuzumab-deruxtecan (TDxD) and an anti-PD-1 antibody on organoid viability.

FIG. 13D provides the results of the combination of TDxD and an anti-PD-1 antibody or trastuzumab and an anti-PD-1 antibody on CD8-expressing cells.

FIG. 13E provides the results of the combination of TDxD and an anti-PD-1 antibody or trastuzumab and an anti-PD-1 antibody on PD-1-expressing cells.

FIG. 13F provides the results of the combination of trastuzumab and an anti-PD-1 antibody on TIM3, B2M, HER2 and PD-L1 expression in the organoid-immune cell co-cultures.

FIG. 14A provides the patient-specific cell lines that were used to generate the organoid-immune cell co-cultures.

FIG. 14B provides the results of the combination of TDxD and an anti-PD-1 antibody or trastuzumab and an anti-PD-1 antibody on patient-specific organoid-immune cell co-cultures shown in FIG. 14A.

FIGS. 14C-14E provide the results of the combination of TDxD and an anti-PD-1 antibody or trastuzumab and an anti-PD-1 antibody on the viability of patient-specific organoid-immune cell co-cultures.

FIG. 15A provides an exemplary protocol for performing fluorouracil-labeled RNA sequencing (Flura-seq).

FIGS. 15B-15E provides the qPCR results for ß-actin (FIG. 15B), GADPH (FIG. 15C), EpCAM (FIG. 15D) and CD3 (FIG. 15E) expression.

FIG. 16 provides a schematic of an organoid-bacterial cell co-culture.

FIG. 17 provides an exemplary protocol for generating organoid-bacterial cell co-cultures according to the present disclosure.

FIG. 18 provides an exemplary organoid-bacterial cell co-culture comprising pks+ E. coli or Δpks E. coli.

FIG. 19 provides an exemplary organoid-bacterial cell co-culture comprising clbP+ E. coli or cultured in the presence of Aphidicolin (APH).

FIG. 20 provides an exemplary protocol for generating organoid-bacterial cell co-cultures according to the present disclosure.

FIG. 21 provides an exemplary view of an organoid-bacterial cell co-culture cultured in 0.005 μg/μl gentamycin.

FIG. 22 provides an exemplary view of an organoid-bacterial cell co-culture cultured in 0.5 μg/μl gentamycin.

FIG. 23 provides an exemplary view of an organoid-bacterial cell co-culture cultured in 5.0 μg/μl gentamycin.

FIG. 24 provides an exemplary view of an organoid-bacterial cell co-culture.

### DETAILED DESCRIPTION

For clarity, but not by way of limitation, the detailed description of the presently disclosed subject matter is divided into the following subsections:
I. Definitions;
II. Organoid Co-cultures;
III. Methods of Use;
IV. Kits; and
V. Exemplary Embodiments.

### I. Definitions

The terms used in this specification generally have their ordinary meanings in the art, within the context of this disclosure and in the specific context where each term is used. Certain terms are discussed below, or elsewhere in the specification, to provide additional guidance to the practitioner in describing the compositions and methods of the present disclosure and how to make and use them.

As used herein, the use of the word “a” or “an” when used in conjunction with the term “comprising” in the claims and/or the specification can mean “one,” but it is also consistent with the meaning of “one or more,” “at least one,” and “one or more than one.”

The terms “comprise(s),” “include(s),” “having,” “has,” “can,” “contain(s),” and variants thereof, as used herein, are intended to be open-ended transitional phrases, terms or words that do not preclude additional acts or structures. The present disclosure also contemplates other embodiments “comprising,” “consisting of” and “consisting essentially of,” the embodiments or elements presented herein, whether explicitly set forth or not.

The term “about” or “approximately” means within an acceptable error range for the particular value as determined by one of ordinary skill in the art, which depends in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

An “individual” or “subject” or “patient” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

The terms “primary” or “primary origin,” as used herein in relation to cancer, refers to the organ in the body of the subject where the cancer began (e.g., the colon). The primary origin of a cancer can be identified using methods known in the art, e.g., medical imaging, examination of biopsy samples with immunohistochemistry techniques, and/or gene expression profiling.

As used herein, and as well-understood in the art, “treatment” is an approach for obtaining beneficial or desired results, including clinical results. For purposes of this subject matter, beneficial or desired clinical results include, but are not limited to, alleviation or amelioration of one or more signs or symptoms, diminishment of extent of disease, stabilized (i.e., not worsening) state of disease, prevention of disease, delay or slowing of disease progression, remission of the disease (e.g., cancer) and/or amelioration or palliation of the disease state. The decrease can be at least a 10%, 20%, 30%, 40%, 50%, 60%, 70%, 80%, 90%, 95%, 98% or 99% decrease in severity of complications, signs or symptoms or in likelihood of progression to another grade. “Treatment” can also refer to inhibiting proliferation of a cancer or progression to a higher grade by at least 10%, 20%, 30%, 40%, 50%, 60%, 70%, 80%, 90%, 95%, 98% or 99%.

As used herein, the term “proliferation” refers to an increase in cell number.

As used herein, the term “in vitro” refers to an artificial environment and to processes or reactions that occur within an artificial environment. In vitro environments exemplified, but are not limited to, test tubes and cell cultures.

As used herein, the term “in vivo” refers to the natural environment (e.g., an animal or a cell) and to processes or reactions that occur within a natural.

The terms “detection” or “detecting” include any means of detecting, including direct and indirect detection.

The term “expression vector” is used to denote a nucleic acid molecule that is either linear or circular, into which another nucleic acid sequence fragment of appropriate size can be integrated. Such nucleic acid fragment(s) can include additional segments that provide for transcription of a gene encoded by the nucleic acid sequence fragment. The additional segments can include and are not limited to: promoters, transcription terminators, enhancers, internal ribosome entry sites, untranslated regions, polyadenylation signals, selectable markers, origins of replication and such, as known in the art. Expression vectors are often derived from plasmids, cosmids, and viral vectors; vectors are often recombinant molecules containing nucleic acid sequences from several sources.

The term “expression” or “express,” as used herein, refers to the transcription and/or translation of a nucleotide sequence.

The term “operably linked,” when applied to nucleic acid sequences, for example in an expression vector, indicates that the sequences are arranged so that they function cooperatively in order to achieve their intended purposes, i.e., a promoter sequence allows for initiation of transcription that proceeds through a linked coding sequence as far as the termination signal.

A “nucleic acid molecule” is a single or double stranded covalently-linked sequence of nucleotides in which the 3′ and 5′ ends on each nucleotide are joined by phosphodiester bonds. The polynucleotide can be made up of deoxyribonucleotide bases or ribonucleotide bases. Polynucleotides include DNA and RNA, and can be manufactured synthetically in vitro or isolated from natural sources.

The term “promoter” as used herein denotes a region within a gene to which transcription factors and/or RNA polymerase can bind so as to control expression of an associated coding sequence. Promoters are commonly, but not always, located in the 5′ non-coding regions of genes, upstream of the translation initiation codon. The promoter region of a gene can include one or more consensus sequences that act as recognizable binding sites for sequence specific nucleic acid binding domains of nucleic acid binding proteins. Nevertheless, such binding sites can also be located in regions outside of the promoter, for example in enhancer regions located in introns or downstream of the coding sequence.

The term “organoid,” as used herein, refers to a three-dimensional cellular structure obtained by expansion of stem cells, e.g., adult stem cells, that self-organize and differentiate into functional cell types. See Corro et al., Am. J. Physiol. Cell Physiol. 319:C151-C165 (2020).

The term “co-culture,” as used herein, refers to a culture of two or more cell types. In certain embodiments, the two or more cell types are maintained in conditions suitable for their growth.

In certain embodiments, an “organoid co-culture” refers to an organoid that is cultured with a different cell type. For example, but not by way of limitation, an organoid co-culture can refer to an organoid cultured with an immune cell or a bacterial cell.

As used herein, the term “population of cells” or “cell population” refers to a group of at least two cells. In certain non-limiting examples, a cell population can include at least about 10, at least about 100, at least about 200, at least about 300, at least about 400, at least about 500, at least about 600, at least about 700, at least about 800, at least about 900, at least about 1000 cells, at least about 5,000 cells or at least about 10,000 cells or at least about 100,000 cells or at least about 1,000,000 cells. The population can be a pure population comprising one cell type, such as a population of cancer cells. Alternatively, the population may comprise more than one cell type, for example a mixed cell population. In certain embodiments, a cell population can include one cell type, where one or more cells within the cell population is a cell derived from a tumor a patient, and one or more other cells within the cell population is an immune cell or a bacterial cell.

As used herein, the term “culture medium” refers to a liquid that covers cells in a culture vessel, such as a Petri plate, a multi-well plate, and the like, and contains nutrients to nourish and support the cells. Culture medium can also include growth factors added to produce desired changes in the cells.

As used herein, the term “dose” refers to a specified quantity of a therapeutic agent. In certain embodiments, a dose can be provided to a subject in a single administration or administered in two or more administrations, e.g., in a single day.

“Immunotherapy” refers to a treatment that induces, suppresses or enhances the immune system of a patient. In certain embodiments, immunotherapies can activate a subject's innate and/or adaptive immune responses to more effectively treat target a disease or disorder, such as cancer.

As used herein, the term “contacting” cells with a compound (e.g., one or more inhibitors, activators and/or inducers) refers to exposing cells to a compound, for example, placing the compound in a location that will allow it to touch the cell. The contacting can be accomplished using any suitable methods. For example, contacting can be accomplished by adding the compound to a tube of cells. Contacting can also be accomplished by adding the compound to a culture medium comprising the cells. Each of the compounds (e.g., the inhibitors, activators and/or inducers) can be added to a culture medium comprising the cells as a solution (e.g., a concentrated solution). Alternatively or additionally, the compounds (e.g., the inhibitors, activators, and inducers disclosed herein) as well as the cells can be in a formulated cell culture medium. In certain embodiments, “contacting” refers to exposing a cell, e.g., a cell within an organoid or in an organoid co-culture, to an agent or compound. In certain embodiments, “contacting” refers to the exposure of organoid or an organoid co-culture to a potential therapeutic agent or therapeutic agent of interest.

As used herein, the term “derived from” or “established from” or “differentiated from” when made in reference to any cell disclosed herein refers to a cell that was obtained from (e.g., isolated, purified, etc.) a parent cell in a cell line, tissue (such as a dissociated tumor) or fluids using any manipulation, such as, without limitation, single cell isolation, cultured in vitro, treatment and/or mutagenesis using for example proteins, chemicals, radiation, infection with virus, transfection with DNA sequences, such as with a morphogen, etc., selection (such as by serial culture) of any cell that is contained in cultured parent cells. A derived cell can be selected from a mixed population by virtue of response to a growth factor, cytokine, selected progression of cytokine treatments, adhesiveness, lack of adhesiveness, sorting procedure and the like.

### II. Organoid Co-Cultures

The present disclosure provides methods of producing organoids and organoid co-cultures. In certain embodiments, the organoids are generated from cells obtained from a subject. In certain embodiments, the organoids are generated from cancer cells obtained from a subject. In certain embodiments, the organoids are generated from normal cells, e.g., epithelial cells, obtained from a subject. In certain embodiments, the subject is a human. In certain embodiments, the subject is a mouse.

In certain embodiments, the present disclosure provides for an organoid co-culture comprising an organoid cultured with a second cell type. For example, but not by way of limitation, organoid co-cultures can be produced by culturing a second cell type with the organoids. In certain embodiments, the second cell type can be derived from the same subject from which the cells of the organoid are derived or obtained. In certain embodiments, the second cell type can be one or more immune cells. In certain embodiments, the second cell type can be one or more bacterial cells. In certain embodiments, the organoid co-cultures can include one or more immune cells and/or one or more bacterial cells.

In certain embodiments, the ratio of organoid cells to the second cell type can be from about 20:1 to about 1:20. For example, but not by way of limitation, the ratio of organoid cells to the second cell type can be from about 19:1 to about 1:19, from about 18:1 to about 1:18, from about 17:1 to about 1:17, from about 16:1 to about 1:16, from about 15:1 to about 1:15, from about 14:1 to about 1:14, from about 13:1 to about 1:13, from about 12:1 to about 1:12, from about 11:1 to about 1:11, from about 10:1 to about 1:10, from about 9:1 to about 1:9, from about 8:1 to about 1:8, from about 7:1 to about 1:7, from about 6:1 to about 1:6, from about 5:1 to about 1:5, from about 4:1 to about 1:4, from about 3:1 to about 1:3, from about 2:1 to about 1:2 or about 1:1. In certain embodiments, the ratio of organoid cells to the second cell type is from about 1:1 to about 20:1. In certain embodiments, the ratio of organoid cells to the second cell type is about 20:1. In certain embodiments, the ratio of organoid cells to immune cells is about 10:1. In certain embodiments, the ratio of organoid cells to the second cell type is about 5:1. In certain embodiments, the ratio of organoid cells to the second cell type is about 2:1. In certain embodiments, the ratio of organoid to the second cell type is about 1:1. In certain embodiments, the ratio of the second cell type to organoid cells is about 20:1. In certain embodiments, the ratio of the second cell type to organoids is about 10:1. In certain embodiments, the ratio of the second cell type to organoid cells is about 5:1. In certain embodiments, the ratio of the second cell type to organoid cells is about 2:1. In certain embodiments, the ratio of the second cell type to organoid cells is about 1:1.

In certain embodiments, a co-culture of the present disclosure can further include any other pathogens, microbes, compounds and/or molecules that can be incorporated in the lumen of an organoid. For example, but not by way of limitation, the co-culture can include a parasite, a virus or a fungal cell and/or molecules including bile acids, metabolites, dietary components and small molecule, e.g., pharmaceutical drugs.

In certain embodiments, an organoid for use in the present disclosure can be generated from normal (i.e., non-cancerous) cells, e.g., epithelial cells. In certain embodiments, any epithelial cell can be used to generate an organoid for use in an organoid co-culture. For example, but not by way of limitation, the normal cells can be skin cells, liver cells, breast cells, lung cells, intestinal cells, renal cells, colon cells or pancreatic cells.

In certain embodiments, an organoid for use in the present disclosure can be generated from cells of a cancer. In certain embodiments, the organoid can be a subject-derived tumor organoid. In certain embodiments, a subject-derived tumor organoid is generated from tumor cells obtained from a subject. In certain embodiments, such organoids can be used to recapitulate the tumor microenvironment of the subject. For example, but not by way of limitation, the organoid can be generated from one or more cells from the cancer of the subject and additional cell types obtained from the subject can be cultured with the organoids to mimic the tumor microenvironment.

In certain embodiments, cells of any type of cancer can be used to generate an organoid for use in an organoid co-culture. In certain embodiments, the cancer can be acute lymphoblastic leukemia, acute myelogenous leukemia, biliary cancer, breast cancer, cervical cancer, chronic lymphocytic leukemia, chronic myelogenous leukemia, colorectal cancer, endometrial cancer, esophagogastric, esophageal, gastric, head and neck cancer, Hodgkin's lymphoma, lung cancer, medullary thyroid cancer, non-Hodgkin's lymphoma, multiple myeloma, renal cancer, ovarian cancer, pancreatic cancer, glioma, melanoma, liver cancer, prostate cancer, and urinary bladder cancer, CD19 malignancies, and other B cell-related or hematologic malignancies. Non-limiting examples of B-cell malignancies include acute lymphoblastic leukemia (ALL), chronic lymphoblastic leukemia (CLL) or non-Hodgkin lymphoma (NHL)). In certain embodiments, the cancer is colorectal cancer. In certain embodiments, the cancer is esophagogastric cancer.

In certain embodiments, the present disclosure provides for an organoid co-culture comprising an organoid culture formed of cancer cells that express LICAM and a second cell type. In certain embodiments, the cancer cells express high levels of LICAM, e.g., as compared to normal or non-cancerous cells. In certain embodiments, the organoid co-cultures can include an organoid expressing high levels of LICAM and a second cell type. In certain embodiments, the organoid co-cultures can include an organoid expressing high levels of LICAM and one or more immune cells and/or one or more bacterial cells.

In certain embodiments, the method for producing an organoid can include obtaining cells for a subject. In certain embodiments, the cells are cancer cells. In certain embodiments, the method can include culturing the cells to generate organoids. For example, but not by way of limitation, cancer cells obtained from a subject can be cultured under conditions that promote organoid formation. In certain embodiments, the method further includes plating the organoids in an extracellular matrix. In certain embodiments, the organoids can be generated using the method described in Example 1.

In certain embodiments, methods of generating organoids co-cultures can include culturing cancer cells and the second cell type in a three-dimension (3D) scaffold. In certain embodiments, methods of generating organoids co-cultures can include culturing cancer cells and the second cell type in an extracellular matrix. In certain embodiments, methods of generating the organoids and culturing the organoids and second cell type in a basement membrane extract (BME). In certain embodiments, the organoids are cultured in a 50% basement membrane extract. In certain embodiments, the ECM for use in the present disclosure includes one or more glycoprotein such as but not limited to collagen, fibronectin and laminin. In certain embodiments, the extracellular matrix can be a commercially available extracellular matrix such as Matrigel™ (BD Biosciences). In certain embodiments, the organoids co-cultures can be generated using the methods described in Examples 1 and 2.

### Organoid-Immune Cell Co-Cultures

The present disclosure provides organoid co-cultures that include one or more immune cells, e.g., one or more immune cell types. In certain embodiments, the co-culture comprises an organoid and an immune cell derived from the same subject. In certain embodiments, such co-cultures can be used to analyzing the effectiveness and/or safety of a therapy for treating the subject.

In certain embodiments, the present disclosure provided methods for generating an organoid-immune cell co-culture. In certain embodiments, the method includes culturing cancer cells e.g., colorectal or esophagogastric cancer cells, in an extracellular matrix to obtain an organoid. In certain embodiments, the method includes culturing normal cells, e.g., normal colon cells, in an extracellular matrix to obtain an organoid. In certain embodiments, the organoids are cultured in media containing Wnt-3a (e.g., from about 1 to about 500 ng/ml), Noggin (e.g., from about 1 to about 500 ng/ml), EGF (e.g., from about 1 to about 500 ng/ml) and/or R-spondin (e.g., from about 0.01 to about 100 ng/ml). In certain embodiments, the organoids are cultured in media containing IFN-γ for about 12 to about 24 hours. In certain embodiments, the organoids can be generated using the method described in Example 1.

In certain embodiments, the method further includes obtaining immune cells and activating the immune cells. In certain embodiments, the immune cells, e.g., T cells, can be activated with an anti-PD-1 antibody. In certain embodiments, the one or more immune cells can be co-cultured with the organoids in an extracellular matrix to generate an organoid-immune cell co-culture. In certain embodiments, the organoid-immune cell co-culture is cultured in media containing Wnt-3a (e.g., from about 1 to about 500 ng/ml), Noggin (e.g., from about 1 to about 500 ng/ml), EGF (e.g., from about 1 to about 500 ng/ml) and/or R-spondin (e.g., from about 0.01 to about 1,000 ng/ml). In certain embodiments, the organoid-immune cell co-cultures can be generated using the method described in Example 1.

In certain embodiments, the organoid-immune cell co-cultures are cultured in media containing Wnt-3a from about 1 to about 500 ng/ml. For example, but not by way of limitation, the organoid-immune cell co-cultures are cultured in media containing Wnt-3a from about 1 to about 450 ng/ml, from about 1 to about 400 ng/ml, from about 1 to about 350 ng/ml, from about 1 to about 300 ng/ml, from about 1 to about 250 ng/ml, from about 1 to about 200 ng/ml, from about 1 to about 150 ng/ml, from about 1 to about 100 ng/ml, from about 5 to about 500 ng/ml, from about 10 to about 500 ng/ml, from about 20 to about 500 ng/ml, from about 30 to about 500 ng/ml, from about 40 to about 500 ng/ml, from about 50 to about 500 ng/ml, from about 60 to about 500 ng/ml, from about 70 to about 500 ng/ml, from about 80 to about 500 ng/ml, from about 90 to about 500 ng/ml, from about 100 to about 500 ng/ml, from about 50 to about 400 ng/ml, from about 50 to about 200 ng/ml or from about 50 to about 150 ng/ml. In certain embodiments, the organoid-immune cell co-cultures are cultured in media containing Wnt-3a at a concentration of about 100 ng/ml.

In certain embodiments, the organoid-immune cell co-cultures are cultured in media containing Noggin from about 1 to about 500 ng/ml. For example, but not by way of limitation, the organoid-immune cell co-cultures are cultured in media containing Noggin from about 1 to about 450 ng/ml, from about 1 to about 400 ng/ml, from about 1 to about 350 ng/ml, from about 1 to about 300 ng/ml, from about 1 to about 250 ng/ml, from about 1 to about 200 ng/ml, from about 1 to about 150 ng/ml, from about 1 to about 100 ng/ml, from about 5 to about 500 ng/ml, from about 10 to about 500 ng/ml, from about 20 to about 500 ng/ml, from about 30 to about 500 ng/ml, from about 40 to about 500 ng/ml, from about 50 to about 500 ng/ml, from about 60 to about 500 ng/ml, from about 70 to about 500 ng/ml, from about 80 to about 500 ng/ml, from about 90 to about 500 ng/ml, from about 100 to about 500 ng/ml, from about 25 to about 400 ng/ml, from about 25 to about 200 ng/ml, from about 25 to about 100 ng/ml or from about 25 to about 75 ng/ml. In certain embodiments, the organoid-immune cell co-cultures are cultured in media containing Noggin at a concentration of about 50 ng/ml.

In certain embodiments, the organoid-immune cell co-cultures are cultured in media containing EGF from about 1 to about 500 ng/ml. For example, but not by way of limitation, the organoid-immune cell co-cultures are cultured in media containing EGF from about 1 to about 450 ng/ml, from about 1 to about 400 ng/ml, from about 1 to about 350 ng/ml, from about 1 to about 300 ng/ml, from about 1 to about 250 ng/ml, from about 1 to about 200 ng/ml, from about 1 to about 150 ng/ml, from about 1 to about 100 ng/ml, from about 5 to about 500 ng/ml, from about 10 to about 500 ng/ml, from about 20 to about 500 ng/ml, from about 30 to about 500 ng/ml, from about 40 to about 500 ng/ml, from about 50 to about 500 ng/ml, from about 60 to about 500 ng/ml, from about 70 to about 500 ng/ml, from about 80 to about 500 ng/ml, from about 90 to about 500 ng/ml, from about 100 to about 500 ng/ml, from about 25 to about 400 ng/ml, from about 25 to about 200 ng/ml, from about 25 to about 100 ng/ml or from about 25 to about 75 ng/ml. In certain embodiments, the organoid-immune cell co-cultures are cultured in media containing EGF at a concentration of about 50 ng/ml.

In certain embodiments, the organoid-immune cell co-cultures are cultured in media containing R-Spondin, e.g., R-Spondin-1, from about 1 to about 1,000 ng/ml. For example, but not by way of limitation, the organoid-immune cell co-cultures are cultured in media containing R-Spondin from about 1 to about 950 ng/ml, from about 1 to about 900 ng/ml, from about 1 to about 850 ng/ml, from about 1 to about 800 ng/ml, from about 1 to about 750 ng/ml, from about 1 to about 700 ng/ml, from about 1 to about 650 ng/ml, from about 1 to about 600 ng/ml, from about 1 to about 550 ng/ml, from about 1 to about 500 ng/ml, from about 1 to about 450 ng/ml, from about 1 to about 400 ng/ml, from about 1 to about 350 ng/ml, from about 1 to about 300 ng/ml, from about 1 to about 250 ng/ml, from about 1 to about 200 ng/ml, from about 1 to about 150 ng/ml, from about 1 to about 100 ng/ml, from about 100 to about 1,000 ng/ml, from about 200 to about 1,000 ng/ml, from about 300 to about 1,000 ng/ml, from about 400 to about 1,000 ng/ml, from about 500 to about 1,000 ng/ml, from about 600 to about 1,000 ng/ml, from about 700 to about 1,000 ng/ml, from about 800 to about 1,000 ng/ml, from about 900 to about 1,000 ng/ml, from about 950 to about 1,000 ng/ml or from about 950 to about 1,100 ng/ml. In certain embodiments, the organoid-immune cell co-cultures are cultured in media containing R-Spondin at a concentration from about 950 to about 1,100 ng/ml. In certain embodiments, the organoid-immune cell co-cultures are cultured in media containing R-Spondin at a concentration from about 1 to about 200 ng/ml. In certain embodiments, the organoid-immune cell co-cultures are cultured in media containing R-Spondin at a concentration of about 1,000 ng/ml. In certain embodiments, the organoid-immune cell co-cultures are cultured in media containing R-Spondin at a concentration of about 100 ng/ml.

In certain embodiments, the organoid-immune cell co-culture is cultured in media containing Wnt-3a (e.g., from about 1 to about 500 ng/ml) and R-spondin (e.g., from about 0.01 to about 1,000 ng/ml). In certain embodiments, the organoid-immune cell co-culture is cultured in media containing Noggin (e.g., from about 1 to about 500 ng/ml) and EGF (e.g., from about 1 to about 500 ng/ml). In certain embodiments, the organoid-immune cell co-culture is cultured in media containing about 100 ng/ml Wnt-3a and about 1,000 ng/ml R-spondin-1. In certain embodiments, the organoid-immune cell co-culture is cultured in media containing about 100 ng/ml Wnt-3a and about 500 ng/ml R-spondin-1. In certain embodiments, the organoid-immune cell co-culture is cultured in media containing about 50 ng/ml Noggin and about 50 ng/ml EGF. In certain embodiments, the organoid-immune cell co-culture is cultured in media containing about 100 ng/ml Wnt-3a, about 1,000 ng/ml R-spondin-1, about 50 ng/ml Noggin and about 50 ng/ml EGF. In certain embodiments, the organoid-immune cell co-culture is cultured in media containing about 100 ng/ml Wnt-3a, about 500 ng/ml R-spondin-1, about 50 ng/ml Noggin and about 50 ng/ml EGF.

In certain embodiments, the media can further include one or more of the following components: from about 1 to about 100 nM gastrin, e.g., about 10 nM gastrin; from about 1 to about 100 mM nicotinamide, e.g., about 10 mM nicotinamide; from about 100 to about 1,000 nM A83-01, e.g., about 500 nM A83-01; from about 1 to about 100 μM SB202190, e.g., about 10 μM SB202190; from about 1 to about 100 mM HEPES, e.g., about 10 mM HEPES; from about 1 to about 10 mM glutamine, e.g., about 2 mM glutamine; from about 1 to about 10 mM N-acetylcysteine, e.g., about 2 mM N-acetylcysteine; from about 0.1 to about 10 mM PGE2, e.g., about 1 μM PGE2; from about 1:50 to about 1:200 N2, e.g., about 1:100 N2; from about 1:10 to about 1:100 B27 (without vitamin A), e.g., about 1:50 B27 (without vitamin A); and from about 1 μg/ml to about 200 μg/ml PRIMOCIN®, e.g., about 100 μg/ml PRIMOCIN®.

In certain embodiments, the ratio of organoid to immune cells can be from about 20:1 to about 1:20. For example, but not by way of limitation, the ratio of organoid to immune cells can be from about 19:1 to about 1:19, from about 18:1 to about 1:18, from about 17:1 to about 1:17, from about 16:1 to about 1:16, from about 15:1 to about 1:15, from about 14:1 to about 1:14, from about 13:1 to about 1:13, from about 12:1 to about 1:12, from about 11:1 to about 1:11, from about 10:1 to about 1:10, from about 9:1 to about 1:9, from about 8:1 to about 1:8, from about 7:1 to about 1:7, from about 6:1 to about 1:6, from about 5:1 to about 1:5, from about 4:1 to about 1:4, from about 3:1 to about 1:3, from about 2:1 to about 1:2 or about 1:1. In certain embodiments, the ratio of organoid to immune cells is from about 1:1 to about 20:1. In certain embodiments, the ratio of organoid to immune cells is about 20:1. In certain embodiments, the ratio of organoid to immune cells is about 10:1. In certain embodiments, the ratio of organoid to immune cells is about 5:1. In certain embodiments, the ratio of organoid to immune cells is about 2:1. In certain embodiments, the ratio of organoid to immune cells is about 1:1. In certain embodiments, the ratio of immune cells to organoids is about 20:1. In certain embodiments, the ratio of immune cells to organoids is about 10:1. In certain embodiments, the ratio of immune cells to organoids is about 5:1. In certain embodiments, the ratio of immune cells to organoids is about 2:1. In certain embodiments, the ratio of immune cells to organoids is about 1:1.

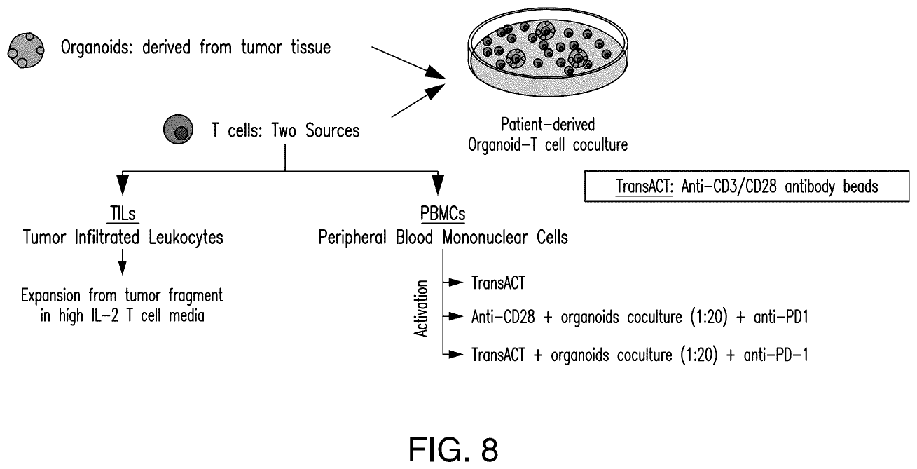

Any immune cell that can be incorporated into a co-culture is suitable for use with methods of the present disclosure. Non-limiting sources of immune cells include peripheral blood mononuclear cells (PBMCs), e.g., isolated from a sample from a subject. In certain embodiments, the PBMCs are isolated from a sample of a subject from which the cells of the organoid are obtained. PBMCs can be isolated from a sample of a subject by any method known in the art, e.g., by a density gradient centrifugation. In certain embodiments, PBMCs are initially isolated from a sample of a donor and then subjected to a selection step to isolate the type of immune cell of interest. In certain embodiments, PBMCs can be obtained using the method described in FIG. 8 and/or Example 1. In certain embodiments, PBMCs can be a PBMC cell line. In certain embodiments, the immune cell can be differentiated from stem cells or iPSC cells.

In certain embodiments, the immune cell is a T cell. For example, but not by way of limitation, the T cell is a CD8⁺ T cell, e.g., a CD8⁺ naïve T cell, a central memory T cell or an effector memory T cell. In certain embodiments, the T cell is a CD4⁺ T cell, a natural killer T cell (NKT cell), a regulatory T cell (Treg), a stem cell memory T cell, a lymphoid progenitor cell, a hematopoietic stem cell, a natural killer cell (NK cell) or a dendritic cell. In certain embodiments, the immune cell is a T cell derived from an iPS cell or a stem cell, e.g., CD8-T cells, CD4+ T cells or CD4+ CD8− T cells, that are differentiated from stem cells or iPSC cells.

In certain embodiments, the immune cell is a tumor infiltrated leukocyte (TIL). For example, but not by way of limitation, TILs for use in the present disclosure can be obtained using the method described in FIG. 8 and/or Example 1. For example, but not by way of limitation, TILs can be isolated from tumor samples of a subject using an antibody, e.g., an anti-CD45 antibody, and optionally followed by expansion in media containing one or more cytokines, e.g., IL-2.

In certain embodiments, the immune cells in the co-culture are engineered T cells. In certain embodiments, the immune cells in the co-culture are engineered to express an antigen-binding receptor. In certain embodiments, the antigen binding receptor is a chimeric antigen receptor (CAR). In certain embodiments, the immune cell is a T cell expressing a CAR (i.e., a CAR T cell). For example, but not by way of limitation, a nucleic acid encoding a CAR is introduced into a T cell, e.g., derived from the subject. In certain embodiments, an expression vector comprising a nucleic acid encoding a CAR that is operably linked to a promoter is introduced into a T cell, e.g., derived from the subject. Any CAR T cell therapy known in the art can be used with the presently disclosed subject matter. In certain embodiments, the CAR T cell can be a CAR T cell comprising an extracellular binding domain that binds to LICAM, mucin 16 (MUC16), B-cell maturation antigen (BCMA), CD19, mesothelin, guanylyl cyclase c (GCC) or a combination thereof. In certain embodiments, the CAR T cell is a LICAM CAR T cell.

In certain embodiments, the immune cells for use in an organoid-immune co-culture are allogeneic with the cells of the organoid. For example, but not by way of limitation, the immune cells are obtained from the same subject as the cells of the organoid. In certain embodiments, the immune cells have been genetically engineered to be allogeneic. In certain embodiments, the immune cells are HLA-matched with the cells of the organoid.

### Organoid-Bacterial Cell Co-Cultures

The present disclosure provides organoid co-cultures that include one or more bacterial cells, e.g., one or more bacterial species. In certain embodiments, the co-culture comprises an organoid and a bacterial species. In certain embodiments, such co-cultures can be used to analyzing the effectiveness and/or safety of a therapy for treating a subject. In certain embodiments, such co-cultures can be used to analyzing the virulence of such bacteria, e.g., the ability of such bacteria to cause carcinogenesis. In certain embodiments, such co-cultures can be used to analyzing the effectiveness of a bacteria to improve the effectiveness of a therapeutic agent.

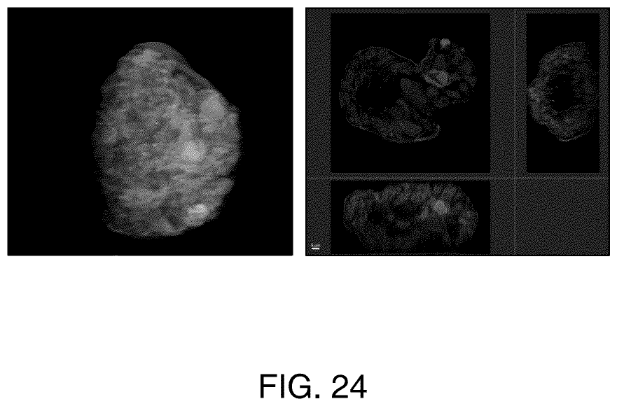

In certain embodiments, an organoid-bacterial cell co-culture can be generated by culturing organoids in the presence of bacterial cells. In certain embodiments, the organoid-bacterial cell co-cultures can be generated using the method described in Example 2. In certain embodiments, the organoids can be cultured in the presence of bacterial cells for about 1 to about 5 days, e.g., about 2 days. In certain embodiments, the bacterial cells are added to an organoid culture as a suspension. In certain embodiments, the bacterial cells can be added to the organoid culture in amount of 50 to about 500 multiplicity of infection (MOI), e.g., about 100 to about 400 or about 100 to about 200 or about 200 MOI. In certain embodiments, the bacterial cells can be added to the organoid culture in amount of about 100 to about 400 MOI. In certain embodiments, the bacterial cells are internalized and reside with the lumen of the organoids in organoid-bacterial cell co-cultures as shown in FIG. 24.

In certain embodiments, the method can further include plating the organoid-bacterial cells in a 3D scaffold. In certain embodiments, the method can further include plating the organoid-bacterial cells in an ECM. In certain embodiments, the method can further include plating the organoid-bacterial cells in a synthetic ECM. In certain embodiments, the method can further include plating the organoid-bacterial cells in Matrigel. In certain embodiments, the organoid-bacterial cells can be washed prior to plating in the Matrigel, e.g., from about 1 time to about 5 times, e.g., about 2 times.

In certain embodiments, an antibiotic can be added to the organoid-bacterial cells prior to plating in the Matrigel. In certain embodiments, an organoid-bacterial cell co-culture can be cultured in the presence of an antibiotic. In certain embodiments, the antibiotic can be gentamicin. In certain embodiments, the antibiotic can be used at a concentration from about 0.001 μg/μl to about 10 μg/μl, e.g., about 0.005 μg/μl, about 0.5 μg/μl or about 5.0 μg/μl. In certain embodiments, the antibiotic can be used at a concentration from about 0.005 μg/μl to about 5 μg/μl, from about 0.005 μg/μl to about 0.5 μg/μl, from about 0.001 μg/μl to about 1.0 μg/μl, from about 0.001 μg/μl to about 1 μg/μl, from about 0.001 μg/μl to about 0.01 μg/μl, from about 0.01 μg/μl to about 1.0 μg/μl or from about 1 μg/μl to about 10 μg/μl. In certain embodiments, the antibiotic can be used at a concentration of about 0.005 μg/μl. In certain embodiments, the antibiotic can be used at a concentration of about 0.005 μg/μl or less. In certain embodiments, the antibiotic can be used at a concentration of about 0.5 μg/μl. In certain embodiments, the antibiotic can be used at a concentration of about 0.5 μg/μl or less. In certain embodiments, the antibiotic can be used at a concentration of about 5.0 μg/μl. In certain embodiments, the antibiotic can be used at a concentration of about 5.0 μg/μl or less.

In certain embodiments, the organoid-bacterial cell co-culture is cultured in media containing Wnt-3a (e.g., from about 1 to about 500 ng/ml), Noggin (e.g., from about 1 to about 500 ng/ml), EGF (e.g., from about 1 to about 500 ng/ml) and/or R-spondin (e.g., from about 0.01 to about 1,000 ng/ml).

In certain embodiments, the organoid-bacterial cell co-cultures are cultured in media containing Wnt-3a from about 1 to about 500 ng/ml. For example, but not by way of limitation, the organoid-bacterial cell co-cultures are cultured in media containing Wnt-3a from about 1 to about 450 ng/ml, from about 1 to about 400 ng/ml, from about 1 to about 350 ng/ml, from about 1 to about 300 ng/ml, from about 1 to about 250 ng/ml, from about 1 to about 200 ng/ml, from about 1 to about 150 ng/ml, from about 1 to about 100 ng/ml, from about 5 to about 500 ng/ml, from about 10 to about 500 ng/ml, from about 20 to about 500 ng/ml, from about 30 to about 500 ng/ml, from about 40 to about 500 ng/ml, from about 50 to about 500 ng/ml, from about 60 to about 500 ng/ml, from about 70 to about 500 ng/ml, from about 80 to about 500 ng/ml, from about 90 to about 500 ng/ml, from about 100 to about 500 ng/ml, from about 50 to about 400 ng/ml, from about 50 to about 200 ng/ml or from about 50 to about 150 ng/ml. In certain embodiments, the organoid-bacterial cell co-cultures are cultured in media containing Wnt-3a at a concentration of about 100 ng/ml.

In certain embodiments, the organoid-bacterial cell co-cultures are cultured in media containing Noggin from about 1 to about 500 ng/ml. For example, but not by way of limitation, the organoid-bacterial cell co-cultures are cultured in media containing Noggin from about 1 to about 450 ng/ml, from about 1 to about 400 ng/ml, from about 1 to about 350 ng/ml, from about 1 to about 300 ng/ml, from about 1 to about 250 ng/ml, from about 1 to about 200 ng/ml, from about 1 to about 150 ng/ml, from about 1 to about 100 ng/ml, from about 5 to about 500 ng/ml, from about 10 to about 500 ng/ml, from about 20 to about 500 ng/ml, from about 30 to about 500 ng/ml, from about 40 to about 500 ng/ml, from about 50 to about 500 ng/ml, from about 60 to about 500 ng/ml, from about 70 to about 500 ng/ml, from about 80 to about 500 ng/ml, from about 90 to about 500 ng/ml, from about 100 to about 500 ng/ml, from about 25 to about 400 ng/ml, from about 25 to about 200 ng/ml, from about 25 to about 100 ng/ml or from about 25 to about 75 ng/ml. In certain embodiments, the organoid-bacterial cell co-cultures are cultured in media containing Noggin at a concentration of about 50 ng/ml.

In certain embodiments, the organoid-bacterial cell co-cultures are cultured in media containing EGF from about 1 to about 500 ng/ml. For example, but not by way of limitation, the organoid-bacterial cell co-cultures are cultured in media containing EGF from about 1 to about 450 ng/ml, from about 1 to about 400 ng/ml, from about 1 to about 350 ng/ml, from about 1 to about 300 ng/ml, from about 1 to about 250 ng/ml, from about 1 to about 200 ng/ml, from about 1 to about 150 ng/ml, from about 1 to about 100 ng/ml, from about 1 to about 500 ng/ml, from about 10 to about 500 ng/ml, from about 20 to about 500 ng/ml, from about 30 to about 500 ng/ml, from about 40 to about 500 ng/ml, from about 50 to about 500 ng/ml, from about 60 to about 500 ng/ml, from about 70 to about 500 ng/ml, from about 80 to about 500 ng/ml, from about 90 to about 500 ng/ml, from about 100 to about 500 ng/ml, from about 25 to about 400 ng/ml, from about 25 to about 200 ng/ml, from about 25 to about 100 ng/ml or from about 25 to about 75 ng/ml. In certain embodiments, the organoid-bacterial cell co-cultures are cultured in media containing EGF at a concentration of about 50 ng/ml.

In certain embodiments, the organoid-bacterial cell co-cultures are cultured in media containing R-Spondin, e.g., R-Spondin-1, from about 1 to about 1,000 ng/ml. For example, but not by way of limitation, the organoid-bacterial cell co-cultures are cultured in media containing R-Spondin from about 1 to about 950 ng/ml, from about 1 to about 900 ng/ml, from about 1 to about 850 ng/ml, from about 1 to about 800 ng/ml, from about 1 to about 750 ng/ml, from about 1 to about 700 ng/ml, from about 1 to about 650 ng/ml, from about 1 to about 600 ng/ml, from about 1 to about 550 ng/ml, from about 1 to about 500 ng/ml, from about 1 to about 450 ng/ml, from about 1 to about 400 ng/ml, from about 1 to about 350 ng/ml, from about 1 to about 300 ng/ml, from about 1 to about 250 ng/ml, from about 1 to about 200 ng/ml, from about 1 to about 150 ng/ml, from about 1 to about 100 ng/ml, from about 100 to about 1,000 ng/ml, from about 200 to about 1,000 ng/ml, from about 300 to about 1,000 ng/ml, from about 400 to about 1,000 ng/ml, from about 500 to about 1,000 ng/ml, from about 600 to about 1,000 ng/ml, from about 700 to about 1,000 ng/ml, from about 800 to about 1,000 ng/ml, from about 900 to about 1,000 ng/ml, from about 950 to about 1,000 ng/ml or from about 950 to about 1,100 ng/ml. In certain embodiments, the organoid-bacterial cell co-cultures are cultured in media containing R-Spondin at a concentration from about 950 to about 1,100 ng/ml. In certain embodiments, the organoid-bacterial cell co-cultures are cultured in media containing R-Spondin at a concentration of about 1,000 ng/ml.

In certain embodiments, the organoid-bacterial cell co-culture is cultured in media containing Wnt-3a (e.g., from about 1 to about 500 ng/ml) and R-spondin (e.g., from about 0.01 to about 1,000 ng/ml). In certain embodiments, the organoid-bacterial cell co-culture is cultured in media containing Noggin (e.g., from about 1 to about 500 ng/ml) and EGF (e.g., from about 1 to about 500 ng/ml). In certain embodiments, the organoid-bacterial cell co-culture is cultured in media containing about 100 ng/ml Wnt-3a and about 1,000 ng/ml R-spondin-1. In certain embodiments, the organoid-bacterial cell co-culture is cultured in media containing about 100 ng/ml Wnt-3a and about 500 ng/ml R-spondin-1. In certain embodiments, the organoid-bacterial cell co-culture is cultured in media containing about 50 ng/ml Noggin and about 50 ng/ml EGF. In certain embodiments, the organoid-bacterial cell co-culture is cultured in media containing about 100 ng/ml Wnt-3a, about 1,000 ng/ml R-spondin-1, about 50 ng/ml Noggin and about 50 ng/ml EGF. In certain embodiments, the organoid-bacterial cell co-culture is cultured in media containing about 100 ng/ml Wnt-3a, about 500 ng/ml R-spondin-1, about 50 ng/ml Noggin and about 50 ng/ml EGF.

In certain embodiments, the media can further include one or more of the following components: from about 1 to about 100 nM gastrin, e.g., about 10 nM gastrin; from about 1 to about 100 mM nicotinamide, e.g., about 10 mM nicotinamide; from about 100 to about 1,000 nM A83-01, e.g., about 500 nM A83-01; from about 1 to about 100 μM SB202190, e.g., about 10 μM SB202190; from about 1 to about 100 mM HEPES, e.g., about 10 mM HEPES; from about 1 to about 10 mM glutamine, e.g., about 2 mM glutamine; from about 1 to about 10 mM N-acetylcysteine, e.g., about 2 mM N-acetylcysteine; from about 0.1 to about 10 mM PGE2, e.g., about 1 μM PGE2; from about 1:50 to about 1:200 N2, e.g., about 1:100 N2; from about 1:10 to about 1:100 B27 (without vitamin A), e.g., about 1:50 B27 (without vitamin A); and from about 1 to about 100 μM Y27632, e.g., about 10 μM Y27632.

Any bacterial cell can be incorporated into a co-culture of the present disclosure. In certain embodiments, the bacterial cell can be a bacterial species of the commensal microbiota. In certain embodiments, the bacterial species can be of the genera Bacteroides, Fusobacterium, Escherichia, Clostridium, Lactobacillus, Bifidobacterium, Peptococcus, Eubacterium and Veillonella. Additional non-limiting examples of commensal bacteria are disclosed in Xu and Gordon, PNAS 100(19): 10452-10459 (2003) and Neish, Reviews in Basic and Clinical Gastroenterology 136(1):P65-80 (2009), the contents of which are incorporated herein in their entireties. In certain embodiments, the bacterial species can be a species that has been implicated in cancer development and/or progression. For example, but not by way of limitation, the bacterial species can be E. coli, e.g., an E. coli strain that expresses polyketide synthetase (pks). In certain embodiments, the bacterial species can be a species that has been implicated in enhancing the effectiveness of a therapeutic agent, e.g., an immunotherapy, e.g., an immune cell expressing an antigen-binding receptor, e.g., a CAR T cell.

### III. Methods of Use

The present disclosure provides methods of using the disclosed organoid co-cultures. In certain embodiments, the present disclosure provides methods of using the disclosed organoid-immune cell co-cultures. In certain embodiments, the present disclosure provides methods of using the disclosed organoid-bacterial cell co-cultures.

In certain embodiments, the organoid co-cultures of the present disclosure can be used for identifying therapeutic agents that can be effective at treating a disease, e.g., cancer. In certain embodiments, the organoid co-cultures of the present disclosure can be used for identifying modified immune cells, e.g., CAR-expressing immune cells, that can be effective at treating a disease, e.g., cancer. In certain embodiments, the organoid co-cultures of the present disclosure can be used to identify bacteria useful in treating cancer or enhancing and/or reducing the benefits of a therapeutic agent. In certain embodiments, the organoid co-cultures can be used to identify bacteria that can cause carcinogenesis.

Non-limiting examples of therapeutic agents that can be analyzed and/or identified using the disclosed methods include protein-based therapeutics, peptide-based therapeutics, immunotherapeutics (e.g., antibody-based therapeutics and modified immune cells such as CAR T cells), antibody-based therapeutics (e.g., antibodies and antibody drug conjugates), small molecule therapeutics and RNA-based therapeutics, e.g., siRNA and RNAi.

In certain embodiments, characteristics of the organoid co-culture can be analyzed to identify changes to the organoid co-culture. For example, but not by way of limitation, analysis of the co-cultures can include flow cytometry, RNA and protein expression, cytokine and metabolite measurements in organoids, immune cells and media, cell viability and proliferation assays, microscopy to monitor epithelial and immune cell motility, migration, proliferation, subcellular localization of RNA, proteins and organelles, cell stiffness and other assays. Organoids, bacterial cells or immune cells can be labeled with dyes or viruses directing the expression of fluorescent proteins and/or luciferase to enable visualization and quantitation of cells using imaging and bioluminescence assays.

In certain embodiments, the change can be a change in cell viability, cell proliferation, organoid size, morphology, invasiveness into basement membrane, motility, differentiation status, mutational status, karyotype, chromosomal aberrations, RNA expression levels and/or protein expression and modifications thereof, chromatin accessibility, histone modifications and other epigenetic changes, physical properties of the organoid, including permeability of the intestinal epithelial barrier, pH, oxygen tension and concentration of other metabolites in the lumen and in the basal membrane and secreted factors in the basement membrane and/or media, concentrations of cytokines and hormones, drug sensitivity, drug absorption and metabolism pharmacokinetics and pharmacodynamics, force measurements, measurements of interactions between biomolecules within the organoids, bacteria, lumen and media/extracellular matrix and membrane potential.

In certain embodiments, the change can be a change in cell viability.

In certain embodiments, the change can be a change in cell proliferation.

In certain embodiments, the change can be a change in organoid size.

In certain embodiments, the change can be a change in mutational status.

In certain embodiments, the change can be a change in RNA expression levels and/or protein expression levels. For example, but not by way of limitation, the change can be a change in PD-1 expression, a change in TIM3 expression, a change in LAG3 expression, a change in B2M expression, a change in HMGB1 expression, a change in HSPA8 expression, a change in HSPA5 expression, a change in PDIA3 expression and/or a change in NFYC expression.

In certain embodiments, the present disclosure provides methods for identifying a therapeutic agent that can be effective in treating a cancer. In certain embodiments, the method can include (i) providing an organoid co-culture comprising cancer cells, (ii) contacting the organoid co-culture with a therapeutic agent and (iii) detecting one or more changes in the organoid co-culture in the presence of the therapeutic agent compared to organoids that have not been contacted with the therapeutic agent. In certain embodiments, the change is indicative of the effectiveness of the therapeutic agent. In certain embodiments, the change can be a change in cell viability, cell proliferation, organoid size, morphology, invasiveness into basement membrane, motility, differentiation 1 status, mutational status, karyotype, chromosomal aberrations, RNA expression levels and/or protein expression and modifications thereof, chromatin accessibility, histone modifications and other epigenetic changes, physical properties of the organoid, including permeability of the intestinal epithelial barrier, pH, oxygen tension and concentration of other metabolites in the lumen and in the basal membrane and secreted factors in the basement membrane and/or media, concentrations of cytokines and hormones, drug sensitivity, drug absorption and metabolism pharmacokinetics and pharmacodynamics, force measurements, measurements of interactions between biomolecules within the organoids, bacteria, lumen and media/extracellular matrix and membrane potential. In certain embodiments, the change is the cell viability of the organoids, e.g., the cells of the organoid. In certain embodiments, the viability of the organoids in the presence of the therapeutic agent is compared to the viability of organoids that have not been contacted with the therapeutic agent. In certain embodiments, if the viability of the organoids that have been contacted with the therapeutic agent is less than the viability of the organoids that have not been contacted with the therapeutic agent, then the therapeutic agent is likely to be effective in treating cancer. In certain embodiments, if the viability of the organoids that have been contacted with the therapeutic agent is the same or greater than the viability of the organoids that have not been contacted with the therapeutic agent, then the therapeutic agent is less likely to be effective in treating cancer. In certain embodiments, the change is the proliferation of the organoids, e.g., the cells of the organoid. In certain embodiments, if the proliferation, e.g., rate of proliferation, of the organoids that have been contacted with the therapeutic agent is less than the proliferation, e.g., rate of proliferation, of the organoids that have not been contacted with the therapeutic agent, then the therapeutic agent is likely to be effective in treating cancer. In certain embodiments, if the proliferation, e.g., rate of proliferation, of the organoids that have been contacted with the therapeutic agent is the same or greater than the proliferation, e.g., rate of proliferation, of the organoids that have not been contacted with the therapeutic agent, then the therapeutic agent is less likely to be effective in treating cancer.

In certain embodiments, the organoid co-culture includes one or more immune cells and/or one or more bacterial cells. In certain embodiments, the organoid co-culture includes T cells expressing a chimeric antigen receptor (CAR T cells). In certain embodiments, the CAR T cells are derived from T cells obtained from the subject from which the cells of the organoid are obtained. In certain embodiments, methods of the present disclosure can be used to identify therapeutic agents that enhance the activity of the CAR T cells present in the organoid co-culture. In certain embodiments, methods of the present disclosure can be used to identify modified immune cells, e.g., CAR T cells, that are effective for treating a cancer.

In certain embodiments, the present disclosure provides methods for testing the efficacy of a candidate modified immune cell. In certain embodiments, the method includes (a) contacting an organoid with the candidate modified immune cell to generate an organoid-immune cell co-culture, (b) detecting the presence of a change in the organoid-immune cell co-culture that is indicative of the effectiveness of the candidate modified immune cell and (c) identifying the candidate modified immune cell as likely to have a therapeutic effect if the change is detected. In certain embodiments, the modified immune cell is an immune cell genetically engineered to express an antigen-binding receptor. In certain embodiments, the antigen-binding receptor is a CAR, e.g., a CAR specific for LICAM. In certain embodiments, the change is a change in cell viability, cell proliferation, organoid size, morphology, mutational status, epigenetic state, RNA expression levels and/or protein expression. In certain embodiments, the change is a change in cell viability. In certain embodiments, the viability of the organoids in the organoid co-culture is compared to the viability of organoids that have not been cultured with the modified immune cell. In certain embodiments, if the viability of the organoids that have been contacted with the modified immune cell is less than the viability of the organoids that have not been contacted with the modified immune cell, then the modified immune cell is likely to be effective in treating cancer. In certain embodiments, if the viability of the organoids that have been contacted with the modified immune cell is the same or greater than the viability of the organoids that have not been contacted with the modified immune cell, then the modified immune cell is less likely to be effective in treating cancer. In certain embodiments, the change is the proliferation of the organoids, e.g., the cells of the organoid. In certain embodiments, if the proliferation, e.g., rate of proliferation, of the organoids that have been contacted with the modified immune cell is less than the proliferation, e.g., rate of proliferation, of the organoids that have not been contacted with the modified immune cell, then the modified immune cell is likely to be effective in treating cancer. In certain embodiments, if the proliferation, e.g., rate of proliferation, of the organoids that have been contacted with the modified immune cell is the same or greater than the proliferation, e.g., rate of proliferation, of the organoids that have not been contacted with the modified immune cell, then the modified immune cell is less likely to be effective in treating cancer. In certain embodiments, this method can be used to identify specific immune cell therapies that can be used to successfully treat a specific subject. In certain embodiments, the organoid is generated from cancer cells of a subject, e.g., cancer cells that express high levels of LICAM. In certain embodiments, the modified immune cells are derived from the subject. In certain embodiments, the organoid includes one or more colorectal or esophagogastric cancer cells.

In certain embodiments, the present disclosure provides methods for identifying a CAR T cell that can be effective in treating a cancer. In certain embodiments, the method can include (i) generating an organoid co-culture comprising cancer cells and a CAR T cell, (ii) culturing the organoid co-culture and (iii) detecting one or more changes in the organoid in the organoid co-culture compared to the organoids that have not been cultured with the CAR T cell. In certain embodiments, the change is indicative of the effectiveness of the CAR T cell therapy. In certain embodiments, the change can be a change in cell viability, cell proliferation, organoid size, morphology, invasiveness into basement membrane, motility, differentiation status, mutational status, karyotype, chromosomal aberrations, RNA expression levels and/or protein expression and modifications thereof, chromatin accessibility, histone modifications and other epigenetic changes, physical properties of the organoid, including permeability of the intestinal epithelial barrier, pH, oxygen tension and concentration of other metabolites in the lumen and in the basal membrane and secreted factors in the basement membrane and/or media, concentrations of cytokines and hormones, drug sensitivity, drug absorption and metabolism pharmacokinetics and pharmacodynamics, force measurements, measurements of interactions between biomolecules within the organoids, bacteria, lumen and media/extracellular matrix and membrane potential. In certain embodiments, the viability of the organoids in the organoid co-culture is compared to the viability of organoids that have not been cultured with the CAR T cell. In certain embodiments, if the viability of the organoids that have been contacted with the CAR T cell is less than the viability of the organoids that have not been contacted with the CAR T cell, then the CAR T cell is likely to be effective in treating cancer. In certain embodiments, if the viability of the organoids that have been contacted with the CAR T cell is the same or greater than the viability of the organoids that have not been contacted with the CAR T cell, then the CAR T cell is less likely to be effective in treating cancer. In certain embodiments, the change is the proliferation of the organoids, e.g., the cells of the organoid. In certain embodiments, if the proliferation, e.g., rate of proliferation, of the organoids that have been contacted with the CAR T cell is less than the proliferation, e.g., rate of proliferation, of the organoids that have not been contacted with the CAR T cell, then the CAR T cell is likely to be effective in treating cancer. In certain embodiments, if the proliferation, e.g., rate of proliferation, of the organoids that have been contacted with the CAR T cell is the same or greater than the proliferation, e.g., rate of proliferation, of the organoids that have not been contacted with the CAR T cell, then the CAR T cell is less likely to be effective in treating cancer. In certain embodiments, this method can be used to identify specific CAR T cell therapies that can be used to successfully treat a specific subject.

In certain embodiments, the present disclosure provides methods for identifying a bacteria species that can enhance or minimize the effectiveness of a therapeutic agent. In certain embodiments, the method can include (i) providing an organoid-bacterial cell co-culture comprising cancer cells and one or more bacteria, (ii) contacting the organoid-bacterial cell co-culture with a therapeutic agent and (iii) detecting one or more changes in the organoids of the organoid-bacterial cell co-culture in the presence of the therapeutic agent compared to organoids that are not cultured with the bacteria and have been contacted with the therapeutic agent. In certain embodiments, the change can be a change in cell viability, cell proliferation, organoid size, morphology, invasiveness into basement membrane, motility, differentiation status, mutational status, karyotype, chromosomal aberrations, RNA expression levels and/or protein expression and modifications thereof, chromatin accessibility, histone modifications and other epigenetic changes, physical properties of the organoid, including permeability of the intestinal epithelial barrier, pH, oxygen tension and concentration of other metabolites in the lumen and in the basal membrane and secreted factors in the basement membrane and/or media; concentrations of cytokines and hormones, drug sensitivity, drug absorption and metabolism pharmacokinetics and pharmacodynamics, force measurements, measurements of interactions between biomolecules within the organoids, bacteria, lumen and media/extracellular matrix, membrane potential In certain embodiments, the change is the viability of the organoids of the organoid-bacterial cell co-culture in the presence of the therapeutic agent compared to the viability of organoids that are not cultured with the bacteria and have been contacted with the therapeutic agent. In certain embodiments, if the viability of the organoids of the organoid-bacterial cell co-culture is increased in the presence of the therapeutic agent compared to the viability of organoids that are not cultured with the bacteria and have been contacted with the therapeutic agent, then the bacterial species is minimizing the effectiveness of therapeutic agent. Alternatively, if the viability of the organoids of the organoid-bacterial cell co-culture is decreased in the presence of the therapeutic agent compared to the viability of organoids that are not cultured with the bacteria and have been contacted with the therapeutic agent, then the bacterial species is enhancing the effectiveness of therapeutic agent. In certain embodiments, the therapeutic agent is an immunotherapy. In certain embodiments, the therapeutic agent is an immune cell modified to express an antigen-binding receptor, e.g., a CAR. In certain embodiments, the therapeutic agent is a CAR T cell. In certain embodiments, the bacteria species is obtained from the microbiome of the subject to be treated with the CAR T cell.

In certain embodiments, the present disclosure provides methods for identifying a bacteria species that can cause carcinogenesis. In certain embodiments, the method can include (i) providing an organoid-bacterial cell co-culture comprising normal cell and one or more bacteria, (ii) culturing the organoid-bacterial cell co-culture and (iii) detecting one or more changes in the organoid in the presence of the bacteria compared to organoids that have not been co-cultured with the bacteria species. In certain embodiments, the change is indicative of the carcinogenic activity of the bacteria species. In certain embodiments, the change can be a change in cell viability, cell proliferation, organoid size, morphology, invasiveness into basement membrane, motility, differentiation status, mutational status, karyotype, chromosomal aberrations, RNA expression levels and/or protein expression and modifications thereof, chromatin accessibility, histone modifications and other epigenetic changes, physical properties of the organoid, including permeability of the intestinal epithelial barrier, pH, oxygen tension and concentration of other metabolites in the lumen and in the basal membrane and secreted factors in the basement membrane and/or media; concentrations of cytokines and hormones, drug sensitivity, drug absorption and metabolism pharmacokinetics and pharmacodynamics, force measurements, measurements of interactions between biomolecules within the organoids, bacteria, lumen and media/extracellular matrix, membrane potential. In certain embodiments, the change can be the identification of new mutations (e.g., by identifying the mutation signature of the organoids) and/or the amount of DNA damage that occurs in the presence of the bacteria compared to organoids that have not been co-cultured with the bacteria species. In certain embodiments, methods for identifying whether a species that can cause carcinogenesis can include implanting the co-cultured organoids into a mouse model to determine if the organoids result in tumor formation and/or metastasize. In certain embodiments, the organoids are generated from normal cells, e.g., normal epithelial cells. In certain embodiments, the organoids are generated from normal cells obtained from a subject.

### IV. KITS

The present disclosure further provides kits for use in the present disclosure. In certain embodiments, the present disclosure further provides kits for use in the presently disclosed methods. In certain embodiments, the present disclosure provides kits for identifying therapeutic agents that can be effective at treating a disease, e.g., cancer. In certain embodiments, the present disclosure provides kits for identifying bacteria useful in treating cancer or enhancing and/or minimizing the benefits of a therapeutic agent. In certain embodiments, the present disclosure provides kits for identifying bacteria that can cause carcinogenesis.

In certain embodiments, a kit of the present disclosure can include an organoid or an organoid co-culture. In certain embodiments, a kit includes an organoid that is generated from one or more cancer cells. In certain embodiments, an organoid co-culture can include one or more organoids and one or more immune cells and/or one or more bacterial cells. In certain embodiments, a kit of the present disclosure can include an organoid-immune cell co-culture. In certain embodiments, a kit of the present disclosure can include an organoid-bacterial cell co-culture. In certain embodiments, the organoids are provided in a separate container from the one or more immune cells and/or one or more bacterial cells. In certain embodiments, the organoids are provided in an extracellular matrix, e.g., Matrigel. In certain embodiments, the organoids can be provided in a cell culture dish and/or a microtiter plate.

In certain embodiments, a kit of the present disclosure can further include instructions for determining whether a therapeutic agent to be tested will be effective in treating a cancer and/or will be effective in enhancing the effectiveness of a CAR T cell therapy. In certain embodiments, a kit of the present disclosure can include instructions for determining if a bacteria species can be useful in treating cancer or enhancing and/or minimizing the benefits of a therapeutic agent. In certain embodiments, a kit of the present disclosure can include instructions for determining if a bacteria species can cause carcinogenesis.

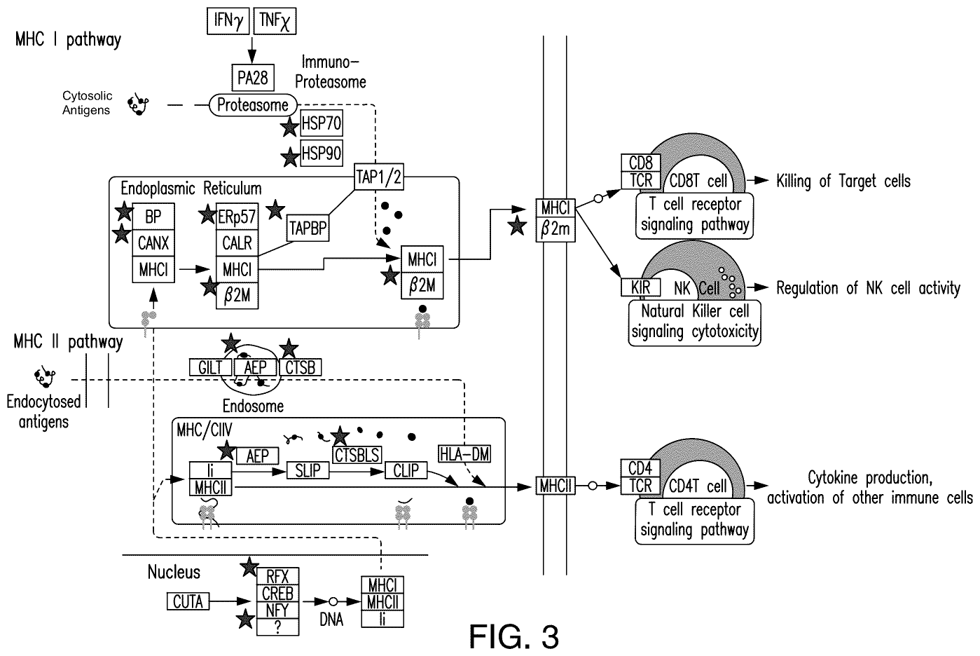

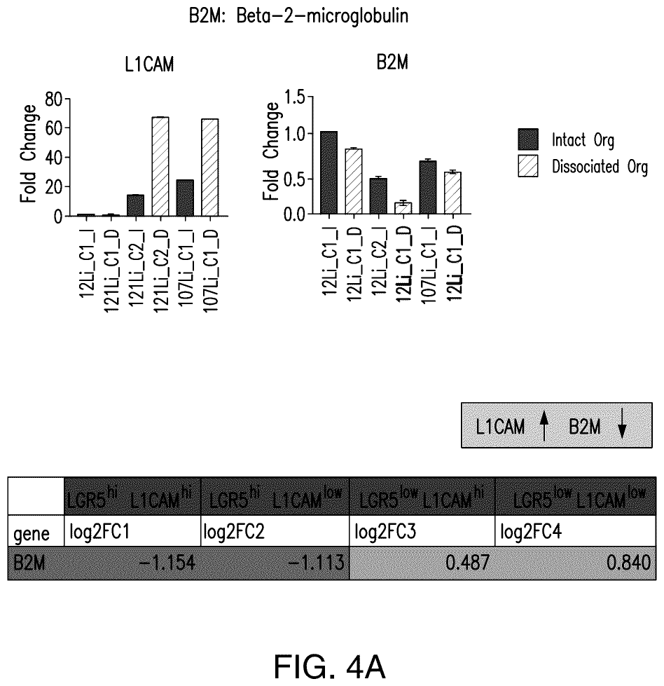

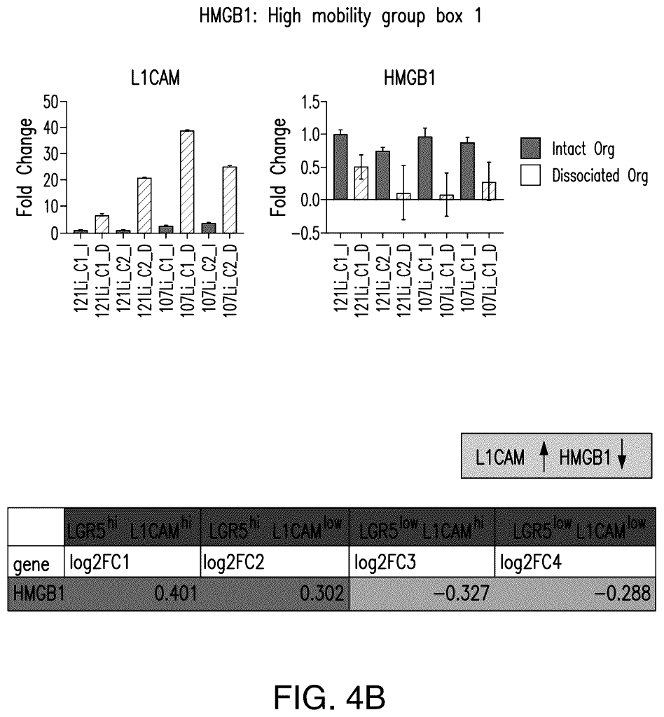

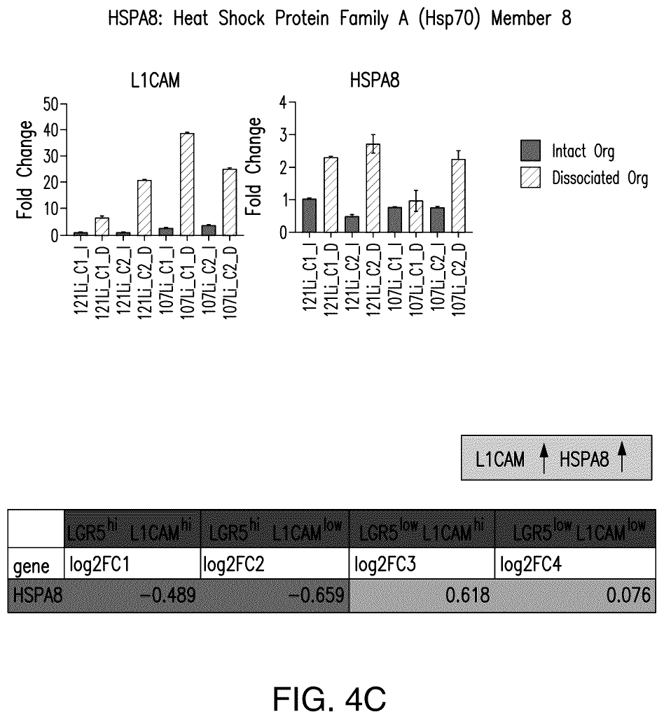

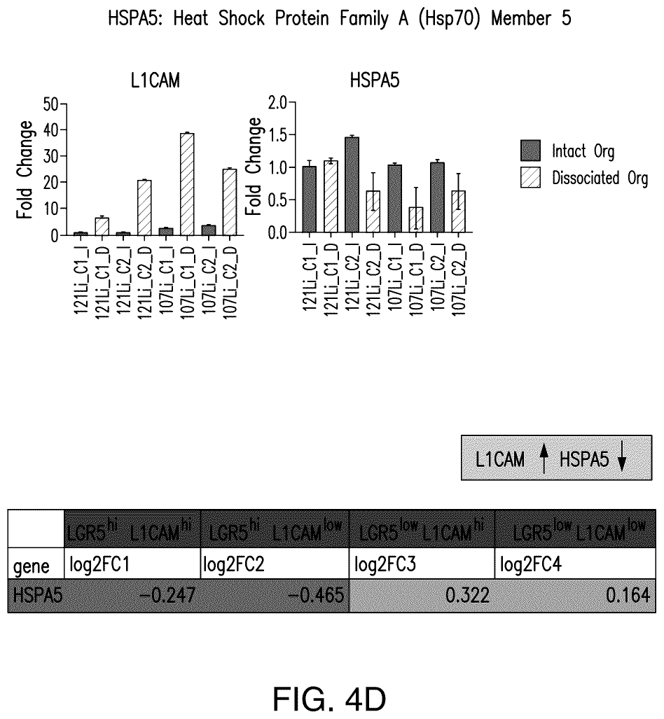

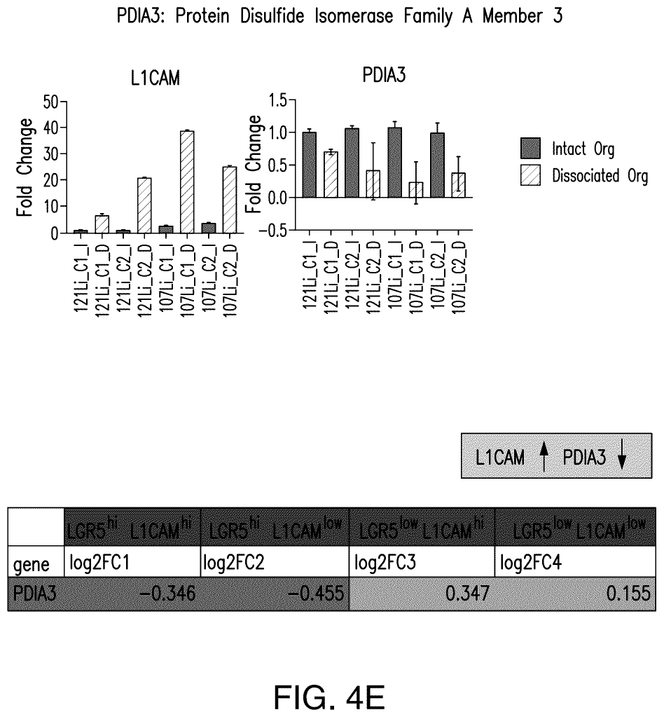

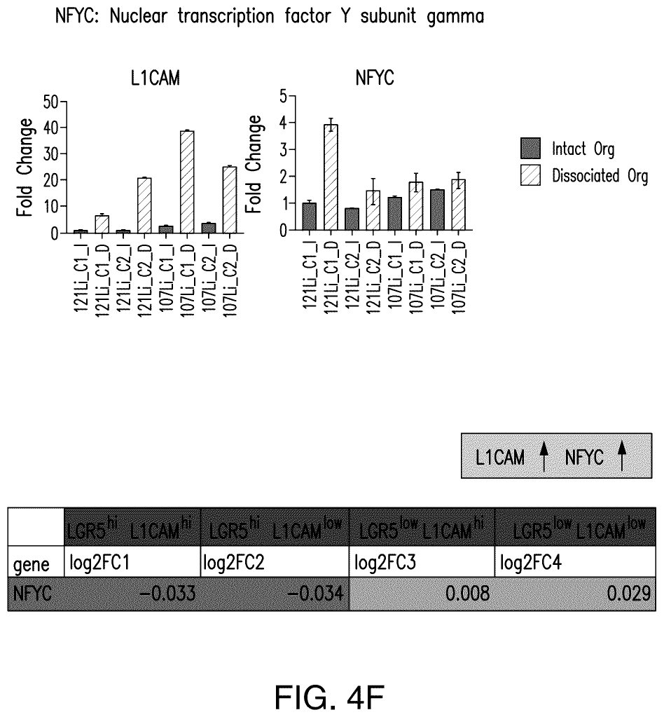

In certain non-limiting embodiments, a kit of the present disclosure can further include one or more reagents and other components (e.g., a buffer, enzymes such as alkaline phosphatase, antibodies, secondary antibodies and the like) to determine the expression of a protein in an organoid co-culture, e.g., the proteins disclosed in FIGS. 3 and 4.

### V. Exemplary Embodiments

A. In certain non-limiting embodiments, the presently disclosed subject matter provides for a method for testing a therapeutic agent that includes: (a) contacting an organoid co-culture with a candidate therapeutic agent; (b) detecting the presence of a change in the organoid co-culture that is indicative of the effectiveness of the therapeutic agent; and (c) identifying the candidate therapeutic agent as likely to have a therapeutic effect if the change is detected.

A1. The method of A, wherein the organoid co-culture comprises an immune cell or a bacterial cell.

A2 The method of A1, wherein the organoid co-culture comprises a bacterial cell.

A3. The method of A1, wherein the organoid co-culture comprises an immune cell.

A4 The method of A3, wherein the immune cell is a T cell.

A5 The method of A4, wherein the T cell is a CAR T cell.

A6. The method of any one of A3-A5, wherein the ratio of the total number cells of the organoid to the total number of immune cells is from about 1:20 to about 20:1, e.g., from about 1:1 to about 20:1.

A7. The method of any one of A-A6, wherein the change is a change in cell viability, cell proliferation, organoid size, morphology, mutational status, epigenetic state, RNA expression levels and/or protein expression.

A8. The method of A7, wherein the change is a change in cell viability.

A9. The method of A8, wherein the change in cell viability is a change in the viability of the cells of the organoid.

A10. The method of any one of A-A9, wherein the organoid is generated from cancer cells of a subject.

All. The method of A10, wherein the immune cells are derived from the subject.

A12. The method of any one of A-A11, wherein the organoid comprises one or more colorectal or esophagogastric cancer cells.

A13. The method of any one of A-A12, wherein the organoid comprises one or cancer cells that express high levels of LICAM.

A14. The method of A5, wherein the CAR T cell is a LICAM CAR T cell.

A15. The method of any one of A-A14, wherein the therapeutic agent is an immunotherapeutic.

A16. The method of A15, wherein the therapeutic agent is an antibody-based therapeutic.

B. In certain non-limiting embodiments, the presently disclosed subject matter provides for a method for testing the efficacy of a candidate modified immune cell comprising: (a) contacting an organoid with the candidate modified immune cell to generate an organoid-immune cell co-culture; (b) detecting the presence of a change in the organoid-immune cell co-culture that is indicative of the effectiveness of the candidate modified immune cell; and (c) identifying the candidate modified immune cell as likely to have a therapeutic effect if the change is detected.

B1. The method of B, wherein the modified immune cell is an immune cell genetically engineered to express an antigen-binding receptor.

B2. The method of B1, wherein antigen-binding receptor is a chimeric antigen receptor (CAR).

B3. The method of any one of B-B2, wherein the change is a change in cell viability, cell proliferation, organoid size, morphology, mutational status, epigenetic state, RNA expression levels and/or protein expression.

B4. The method of B3, wherein the change is a change in cell viability.

B5. The method of any one of B-B4, wherein the organoid is generated from cancer cells of a subject.

B6. The method of B5, wherein modified immune cells are derived from the subject. B7. The method of any one of B-B6, wherein the organoid comprises one or more colorectal or esophagogastric cancer cells.

B8. The method of any one of B-B7, wherein the organoid comprises one or cancer cells that express high levels of LICAM.

B9. The method of any one of B-B8, wherein the modified immune cell is a CAR T cell.

B10. The method of B9, wherein the CAR T cell is a LICAM CAR T cell.

C. In certain non-limiting embodiments, the presently disclosed subject matter provides for a method for analyzing a bacterial species comprising: (a) culturing an organoid with a candidate bacterial species to generate an organoid-bacterial cell co-culture; (b) detecting the presence of a change in the organoid-bacterial cell co-culture that is indicative of the oncogenic potential of the bacterial species; and (c) identifying the bacterial species as likely to have an oncogenic effect if the change is detected.

C1. The method of C, wherein the organoid co-culture further comprises an immune cell.

C2. The method of C1, wherein the immune cell is a T cell.

C3. The method of any one of C-C2, wherein the change is a change in cell viability, cell proliferation, organoid size, morphology, mutational status, epigenetic state, RNA expression levels and/or protein expression.

C4. The method of C3, wherein the change is a change in mutational status.

C5. The method of C4, wherein the change in mutational status is a change in mutational status of one or more cells of the organoid.

C6. The method of any one of C-C5, wherein the organoid is generated from normal cells of a subject.

D. In certain non-limiting embodiments, the presently disclosed subject matter provides for a method for analyzing a bacterial species comprising: (a) providing an organoid-bacterial cell co-culture comprising cancer cells and one or more candidate bacteria; (b) contacting the organoid-bacterial cell co-culture with a therapeutic agent; (c) detecting the presence of a change in the organoid-bacterial cell co-culture that is indicative of the potential of the bacterial species to increase the effectiveness of the therapeutic agent; and (d) identifying the bacterial species as likely to increase the effectiveness of the therapeutic agent if the change is detected.

D1. The method of D, wherein the organoid-bacterial cell co-culture further comprises an immune cell.

D2. The method of D, wherein the therapeutic agent is an immune cell.

D3. The method of D or D1, wherein the immune cell is a T cell.

D4 The method of any one of D-D3, wherein the change is a change in cell viability, cell proliferation, organoid size, morphology, mutational status, epigenetic state, RNA expression levels and/or protein expression.

D5. The method of D4, wherein the change is a change is a change in cell viability.

D6. The method of D5, wherein the change is a change in cell viability is a decrease in the viability of the cells of the organoid.

D7. The method of any one of D-D6, wherein the organoid is generated from cancer cells of a subject.

D8. The method of any one of D2-D7, wherein the immune cell is a modified immune cell.

D9. The method of D8, wherein the modified immune cell is a CAR T cell.

D10. The method of any one of A-D9, wherein the organoid co-culture, the organoid-immune cell co-culture and/or the organoid-bacterial cell co-culture is cultured in a media comprising: (a) from about 1 to about 500 ng/ml of Wnt3a; (b) from about 1 to about 500 ng/ml of Noggin; (c) from about 1 to about 500 ng/ml of EGF; (d) from about 0.01 to about 1,000 ng/ml of R-spondin-1; or (e) a combination of any one of (a)-(d).

D11. The method of D10, wherein the organoid co-culture is cultured in a media comprising about 100 ng/ml Wnt-3a, about 1,000 ng/ml R-spondin-1, about 50 ng/ml Noggin and about 50 ng/ml EGF.

D12. The method of any one of A-D11, wherein the organoid co-culture, the organoid-immune cell co-culture and/or the organoid-bacterial cell co-culture is cultured in a media comprising an antibiotic at a concentration from about 0.005 μg/μl to about 5 μg/μl.

D13. The method of D12, wherein the concentration of the antibiotic is from about 0.001 μg/μl to about 1.0 μg/μl.

D14. The method of D12 or D13, wherein the concentration of the antibiotic is from about 0.005 μg/μl to about 0.5 μg/μl.

E. In certain non-limiting embodiments, the presently disclosed subject matter provides for an organoid co-culture that includes one or more organoid cells and one or more immune cells or bacterial cells, wherein the ratio of organoid cells to immune or bacterial cells is from about 20:1 to about 20:1.

E1. The method of E, wherein the ratio of organoid cells to immune or bacterial cells is from about 1:1 to about 20:1.

E2. The method of E or E1, wherein the ratio of organoid cells to immune or bacterial cells is about 1:10.

E3. The method of any one of E-E2, wherein the ratio of organoid cells to immune or bacterial cells is about 1:5.

E4. The method of any one of E-E3, wherein the ratio of organoid cells to immune or bacterial cells is about 1:2.

F. In certain non-limiting embodiments, the presently disclosed subject matter provides for a kit comprising the organoid co-culture of any one of E-E4.

G. In certain non-limiting embodiments, the presently disclosed subject matter provides for a kit for performing the method of any one of A-D14.

### EXAMPLES

The presently disclosed subject matter will be better understood by reference to the following Examples, which are provided as exemplary of the presently disclosed subject matter, and not by way of limitation.

### Example 1: Formation of Organoids with Immune Cells

This Example discloses the generation of an organoid-immune cell co-culture. In particular, this Example discloses the generation of organoids-CAR T cell co-cultures and organoid-T cell co-cultures.

Normal and tumor colon organoids were generated as follows: Normal colon crypts were isolated with 8 mM EDTA in PBS. Human primary or metastatic tumor samples were grossly dissected, washed, chopped into 5-mm fragments and incubated in dissociation buffer (DMEM with 5% FBS (Gibco), 2 mM 1-glutamine (Fisher Scientific), penicillin-streptomycin (Fisher Scientific), 40 μg/ml gentamicin (Thermo Fisher Scientific), 250 U/ml type III collagenase (Worthington) and 1 U/ml dispase (Sigma-Aldrich)) on a shaker for 30 min at 37° C., filtered through a 70-μm cell strainer (Greiner Bio-One), centrifuged at 600 g for 5 min and washed with ADF wash buffer (Advanced DMEM/F12 containing 2 mM glutamax and 10 mM Hepes). Cells were counted and resuspended in Matrigel at approximately 2,000-10,000 cells per 40 μl of Matrigel in uncoated CELLSTAR multiwell culture plates (Greiner Bio-One). After Matrigel polymerization, HISC medium (Advanced DMEM/F12 containing 100 ng/ml Wnt-3a (R&D; or conditioned medium from L-Wnt3A cells (ATCC)), 1 μg/ml R-Spondin 1 (Peprotech; or conditioned medium from m-RSpo-Fc cells), 50 ng/ml EGF, 50 ng/ml Noggin (Peprotech), 10 nM gastrin (Sigma), 10 mM nicotinamide (Sigma), 500 nM A83-01 (Sigma), 10 μM SB202190, 10 mM HEPES, 2 mM glutamine, 2 mM N-acetylcysteine, 1 μM PGE2 (Sigma), 1:100 N2 (Invitrogen), 1:50 B27 (without vitamin A) and 100 μg/ml Primocin (InvivoGen)) was added. Y27632 (10 μM; Sigma) is added for initial organoid generation and for 48 h after every passage. Mouse tumor and normal colon crypt organoids were generated similarly except that the media used was MISC (Advanced DMEM/F12 containing 2 mM glutamax, 10 mM Hepes, recombinant murine EGF 50 ng/mL, recombinant murine noggin 50 ng/ml, recombinant human R-spondin-1 500 ng/ml or conditioned media, recombinant murine wnt-3a 100 ng/ml or conditioned media and 10 mM nicotinamide).

Immune cells for use in the organoid-immune cell co-cultures were prepared as follows: Tumor/epithelial antigen-specific immune cells were directly expanded from tumors or mucosa by chopping the tissue into 5 mm fragments and plating in media containing IL-2 (for T cells); IL-2 and IL-15 (for NK cells) or other cytokines. In addition, immune cells were expanded from peripheral blood mononuclear cells: Peripheral blood was collected in sodium heparin or BD Vacutainer CPT tubes, mononuclear cells were isolated by density gradient centrifugation, counted and either used fresh or cryopreserved until future use. Tumor or normal epithelial organoids (“target”) were gently dissociated into clumps using TryPle, and cells were counted. Freshly thawed or fresh PBMCs (“effector”) were counted and mixed with organoids with a target:effector ratio of 20:1 in TexMACS media containing appropriate cytokines for specific immune cell expansion. For T cell expansion, 100-3000 IU/ml IL-2 and 40 μg/ml anti-PD1 antibody (Nivolumab) was used. Penicillin/Streptomycin (1:100) and primocin (1:500) was added to prevent bacterial contamination. The resulting suspension was diluted to a concentration of 1-1.5×10⁶ PBMCs/ml and 200 μL of the organoid PBMC mix was plated per well of a U-bottom 96-well plate pre-coated with 2 μL TransACT per well. After 24 hours, cells were harvested, centrifuged at 300-350 g at RT for 5 min, counted and replated in U-bottom 96 well plates at a concentration of 1-1.2×10⁶/ml in TexMACS media containing penicillin/streptomycin at a 1:100 dilution. The cultures were incubated at 37° C. in a 5% CO₂ incubator. The organoid:immune cell suspensions were monitored daily for confluence or change in media color and pH, and passaged upon confluence (typically every alternate day), adding 50% fresh media containing cytokines (100-3000 IU/ml IL-2 for T cell expansion). Immune cells continued to be expanded for 7-14 days (typically 10 days) prior to plating for experimental co-culture. T cells can be reactivated up to twice using TransACT as above for 24-48 hours. Following immune cell expansion, the cell suspension was harvested, flow-sorted using antibodies for the desired cell populations (CD3, CD8 and/or CD4 for T cells) to separate immune cells from the antigen-presenting epithelial cells.

For the co-culture experiments, a fresh population of organoids (either the same line that was used for immune cell activation or a different line from the same or another subject) was used. Organoids were harvested from basement membrane and resuspended in ADF wash buffer, an aliquot of the organoid suspension was dissociated to a single cell suspension using TrypLE and counted. Organoids were seeded either as dissociated single cells or as intact 3-dimensional clusters at a density of 2,000-50,000 cells/40 μl basement membrane extract/well of a 96 well U bottom plate. The basement membrane extract containing organoid suspension was allowed to solidify in a 37° C. in a 5% CO₂ incubator for 15-30 minutes. Immune cells prepared as described above were counted and added to HISC (for human organoids) or MISC (for mouse organoids) media with a target (organoid):effector (immune cells) ratio of 1:1 to 1:20. The resulting co-culture was analyzed at various time points up to 7 days from the start of co-culture.

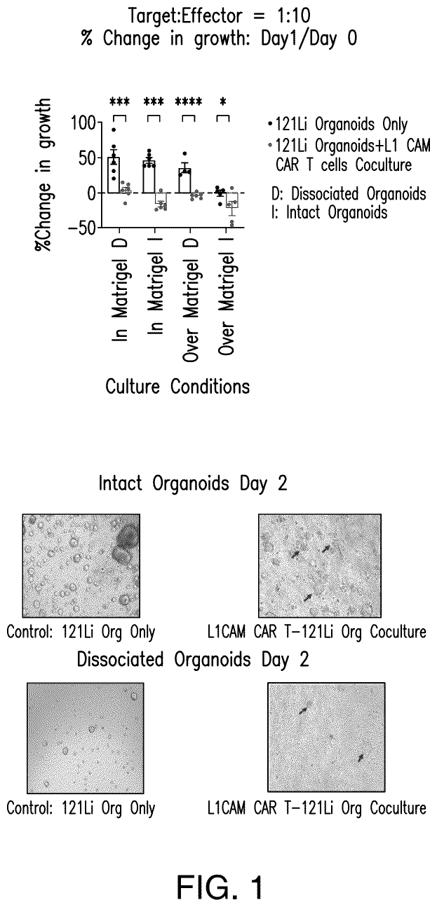

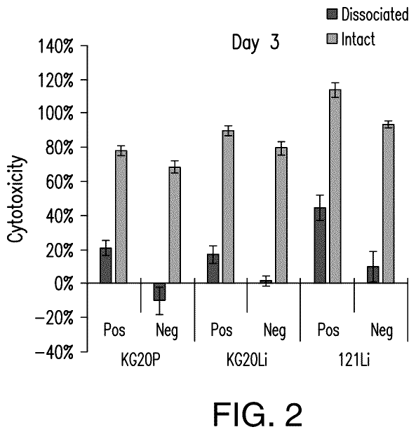

Organoids generated from colorectal cancer cells that were LICAM positive were co-cultured with LICAM CAR T cells (FIGS. 1 and 2). The organoids were either dissociated or kept intact and the effect of the LICAM CAR T cells on the organoids at a target (organoid cells):effector (immune cells) ratio of 1:10 were analyzed. As shown in FIGS. 1 and 2, intact organoids were more successfully killed with LICAM CAR T cells compared to dissociated organoids although LICAM expression is higher in dissociated organoids. These results further shown that the CAR T cells can perform chemotaxis and invade Matrigel.

To determine how dissociated organoids can evade T cell mediated killing, the expression of certain genes was analyzed in intact organoids based on LGR5 and LICAM expression. A number of genes were identified that were either upregulated or downregulated as shown in FIG. 3, including GAPDH, LICAM, B2M, HMGB1, CANX, TAPBP, PDIA3, HSPA8, NFYC, HSPA5, HSPA90AB1, HSPAA1 and LGMN, qPCR was performed on intact (I) and dissociated (D) organoids to compare expression of B2M (FIG. 4A), HMGB1 (FIG. 4B), HSPA8 (FIG. 4C), HSPA5 (FIG. 4D), PDIA3 (FIG. 4E) and NFYC (FIG. 4F). These data show that the disclosed organoid system allows the determination if certain genes are important in the interactions of the immune system with colorectal cancer cells and for escaping the immune system.

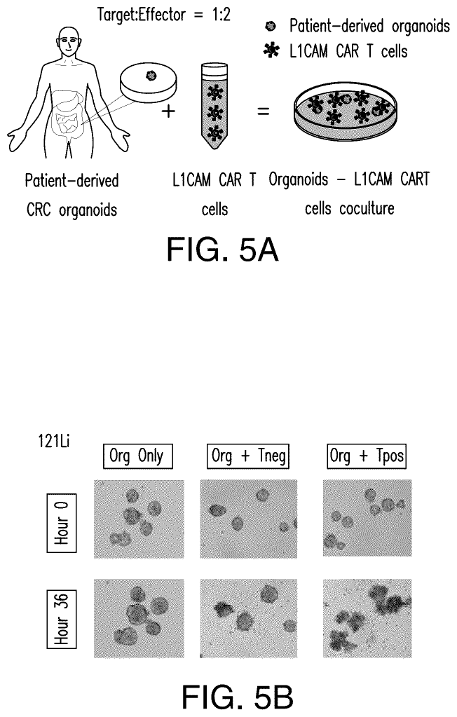

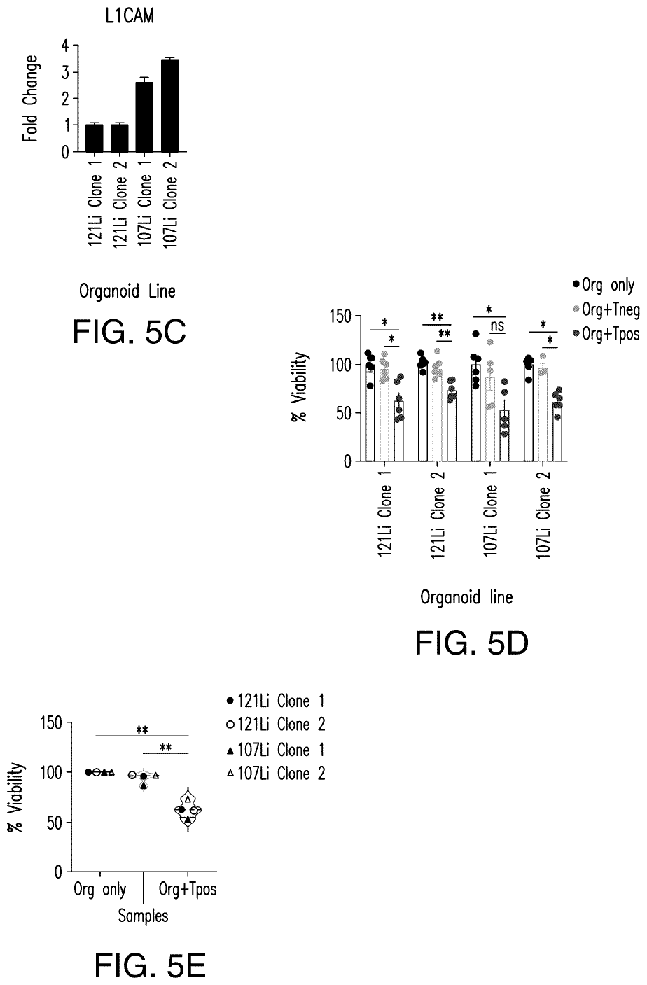

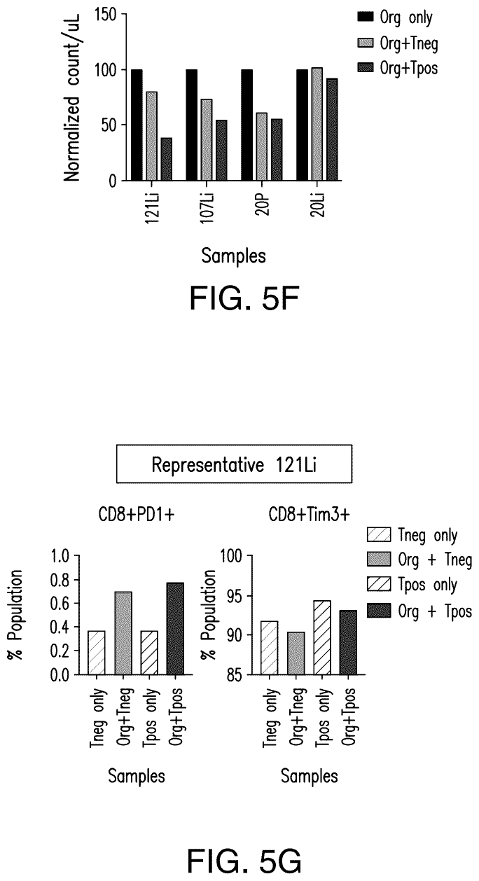

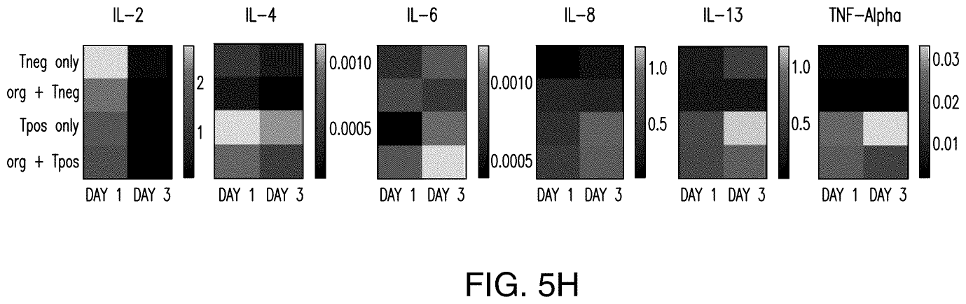

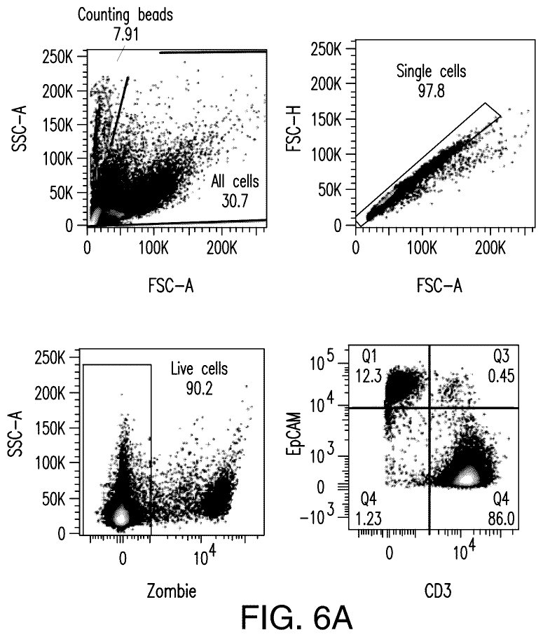

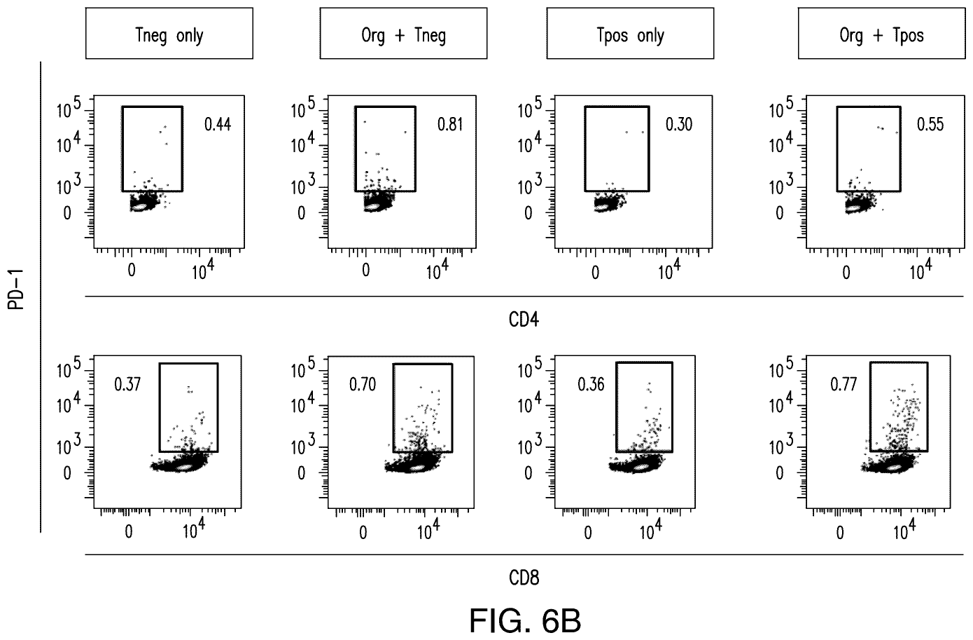

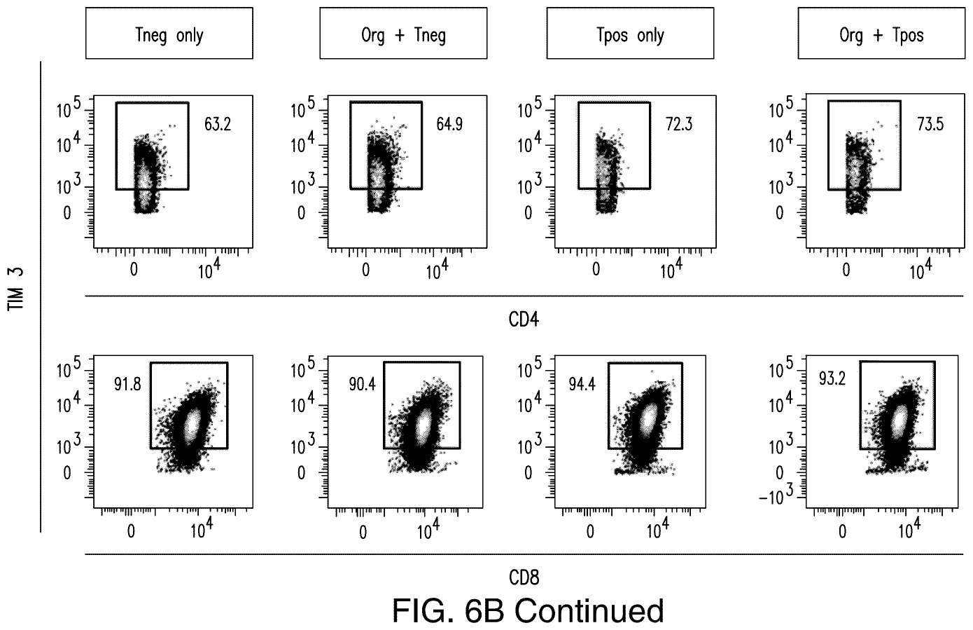

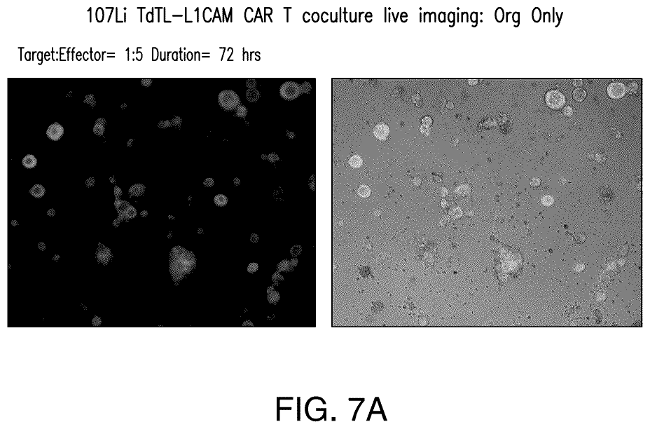

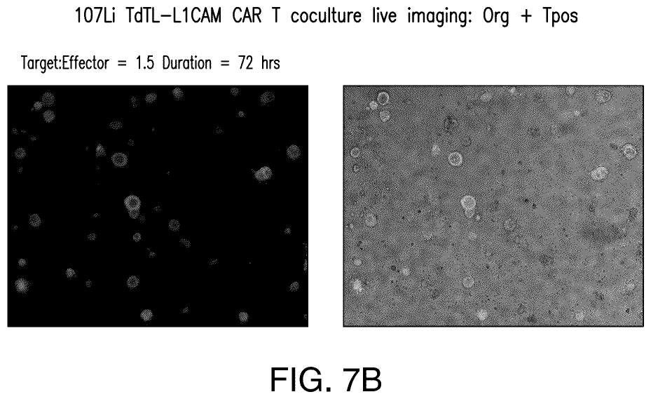

The above experiments led to the identification of a model for an organoid-CAR T cell co-culture as shown in FIG. 5A. The intact organoids were embedded in Matrigel and T cells were added dropwise above it into the media. As shown in FIGS. 5B-5C, LICAM CAR T cells at a target:effector ratio of 1:2 resulted in the killing of organoids compared to organoids cultured without LICAM CAR T cells. A target: effector ratio of 1:5 also led to cell death after 72 hours (FIGS. 7A-7B). As shown in FIGS. 5D-5F and FIGS. 6A-6B, co-culturing LICAM CAR T cells resulted in cytotoxicity of organoids compared to control. In addition, as shown in FIGS. 5G and 6B, PD-1 expression increased in the organoid-CAR T cell co-cultures, but no significant difference was observed TIM3 expression. FIG. 5H shows the expression of cytokines, e.g., IL-2, IL-4, IL-6, IL-8, IL-13 and TNF-alpha expression in the organoid-CAR T cell co-cultures.

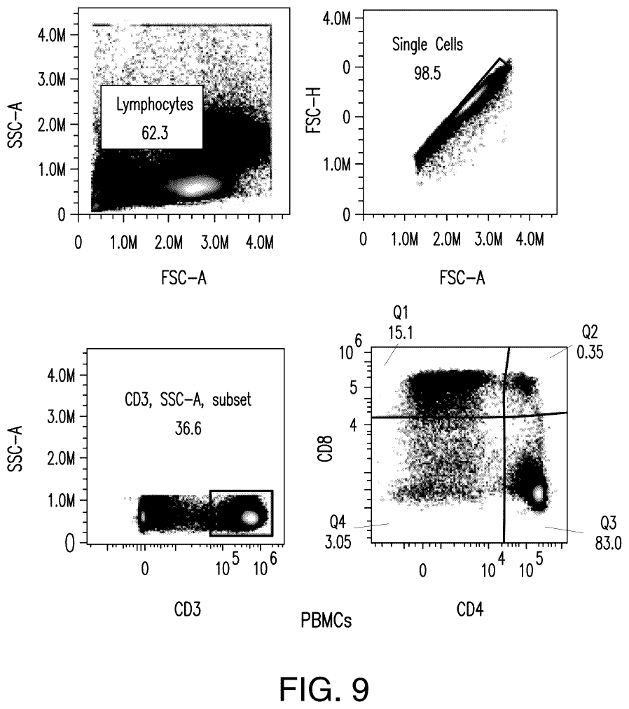

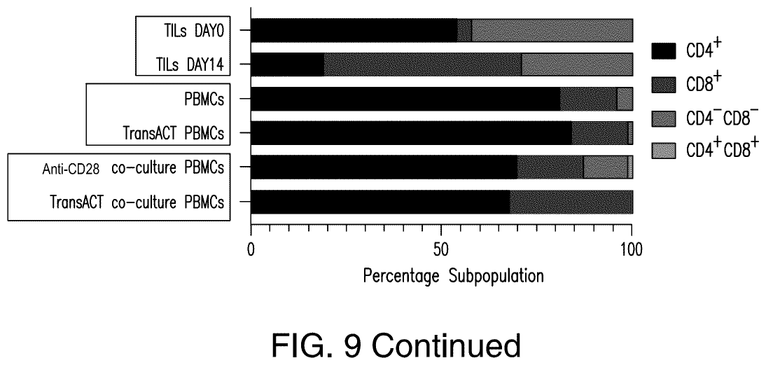

Next, patient specific organoid-T cell co-cultures were generated, where both of the organoids and T cells would be derived from same patient. Two particular sources of T cells were analyzed: tumor infiltrated leukocytes (TILs) and peripheral blood mononuclear cells (PBMCs) (FIG. 8). TILs were directly isolated from tumor samples by using CD45 antibody aided selection through MACS, and expanding the TILs by culturing in T cell media with high IL-2. For PBMCs, they were activated using anti CD3/CD28 conjugated antibody beads which are commercially available as TransAct for T cell expansion from PBMCs. In addition, PBMCs were cocultured with dissociated organoids and anti-PD-1 in anti-CD28 antibody coated wells to provide PBMCs with antigenic stimulation of tumor organoids and expansion of tumor reactive T cells. Further details regarding the isolation of TILs and PBMCs are provided above. As shown in FIG. 9, the isolated TILs mostly included CD4+ T cells, included some cytotoxic CD8+ and a lot of CD8−CD4− cells. As they expanded, the CD4+ population decreased, while the cytotoxic population increased proportionally and the CD8−CD4− slightly decreased. In the isolated PBMCs, many CD4+ cells were detected along with some cytotoxic cells while there was a very low amount of CD8−CD4− cells. This proportion is mostly retained when the T cells are activated with TransACT.

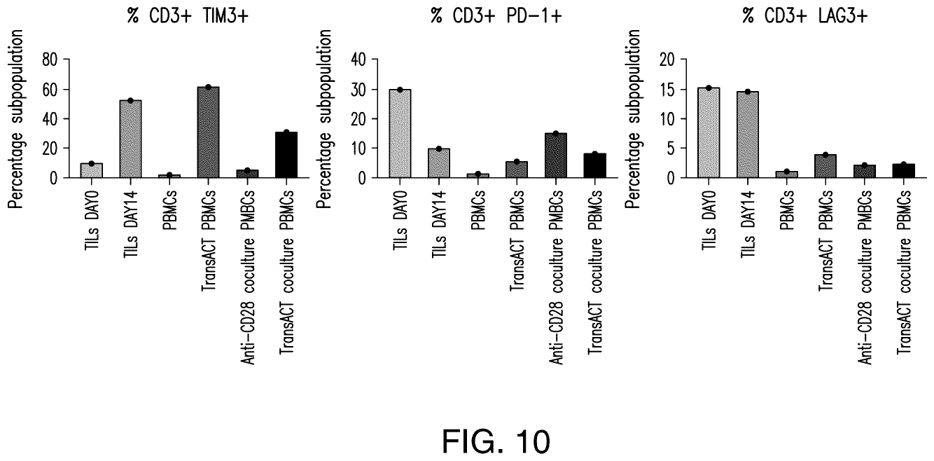

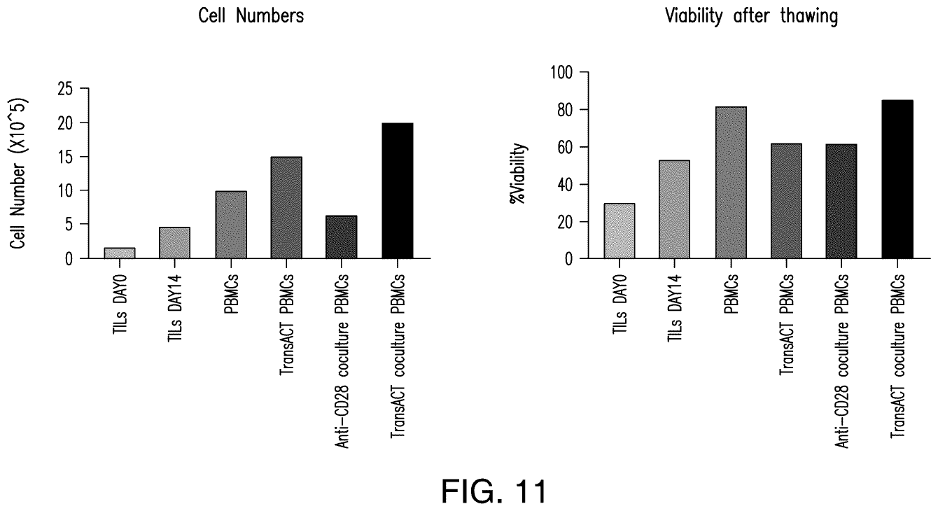

The exhaustion and activation markers expressed in these T cells derived from different sources and time points were also analyzed. As shown in FIG. 10, all three exhaustion and activation markers: TIM3, PD-1 and LAG3 were higher in TILs compared to PBMCs. For TIM3, which is an activation marker, it was observed that the expression goes up as time progresses which is opposite to the trend for immune checkpoint PD-1 expression. This trend is also seen in the PBMCs and the co-cultures. Further, TIM3 expression in TransAct-associated expansion suggests that it activates PBMCs more than anti-CD28 stimulation alone. Further, PD-1 expression appeared to be higher in the co-cultures. With respect to the exhaustion marker LAG3, higher expression is observed in TILs, compared to PBMCs. In addition, PBMCs were observed to have greater viability after thawing (FIG. 11) and greater cell numbers compared to TILs.

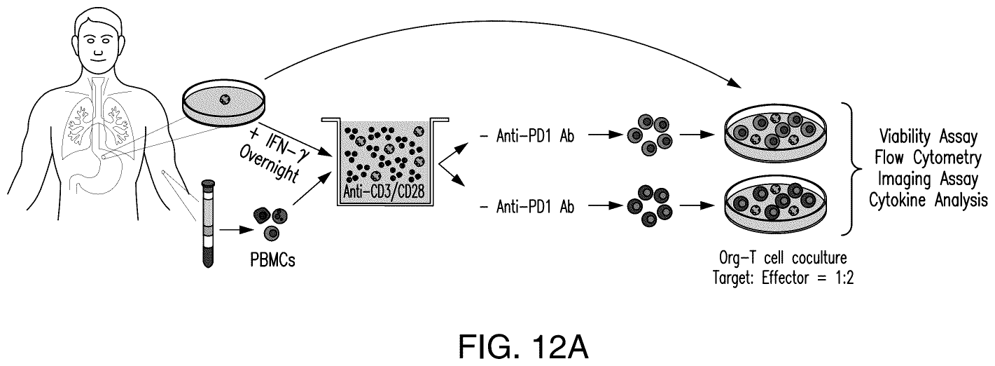

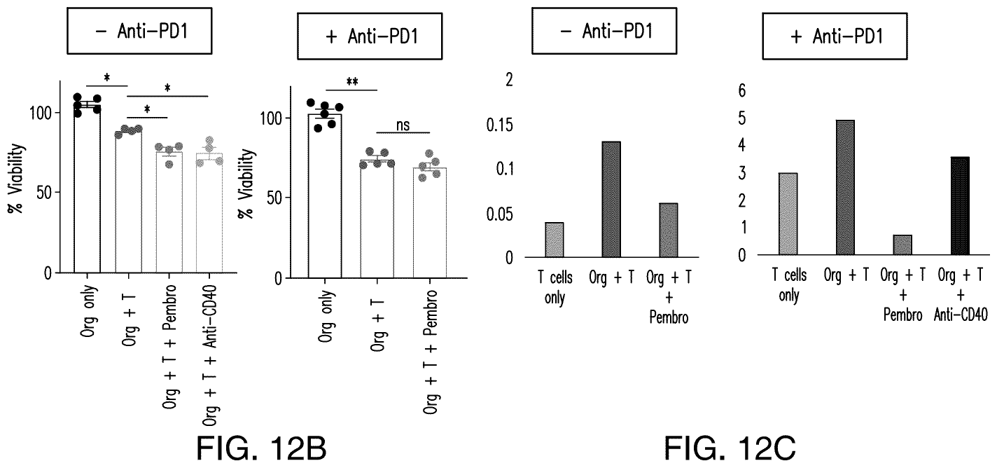

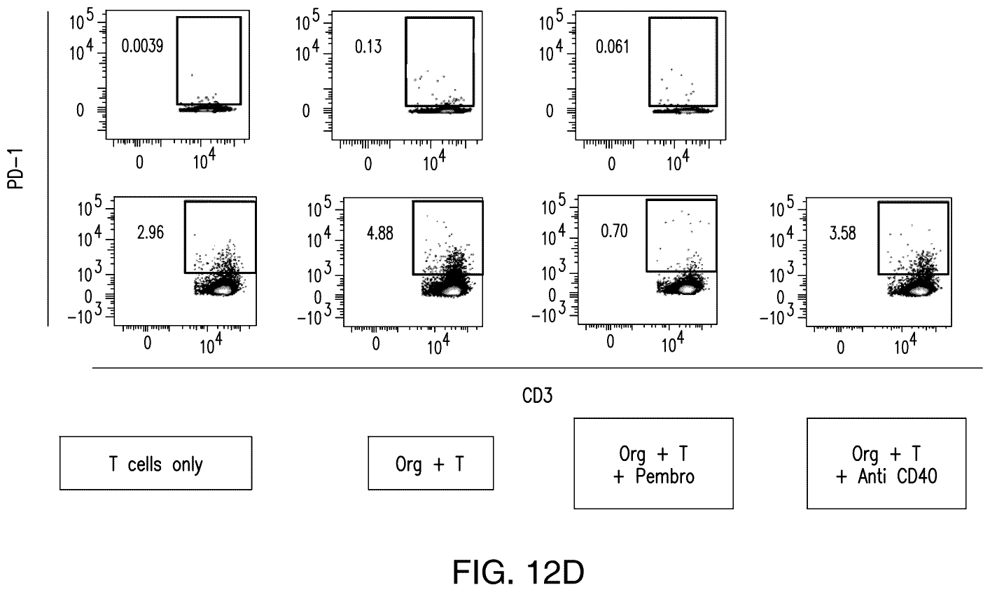

FIG. 12A shows the exemplary experimental design for generating patient-specific organoid-T cell co-cultures, which includes culturing the organoids in IFN-γ overnight and subsequent incubation with PBMCs. This organoid-immune cell co-culture can be used to the efficacy of potential therapeutic regimens. For example, the effect of anti-PD-1 antibody (pembrolizumab) on T cell killing of organoids was tested by contacting the PBMCs and organoids with or without the anti-PD-1 antibody. As shown in FIGS. 12B-12D, contact with the anti-PD-1 antibody resulted in the expansion of the T cells and decreased viability of the organoids in the presence of such T cells.

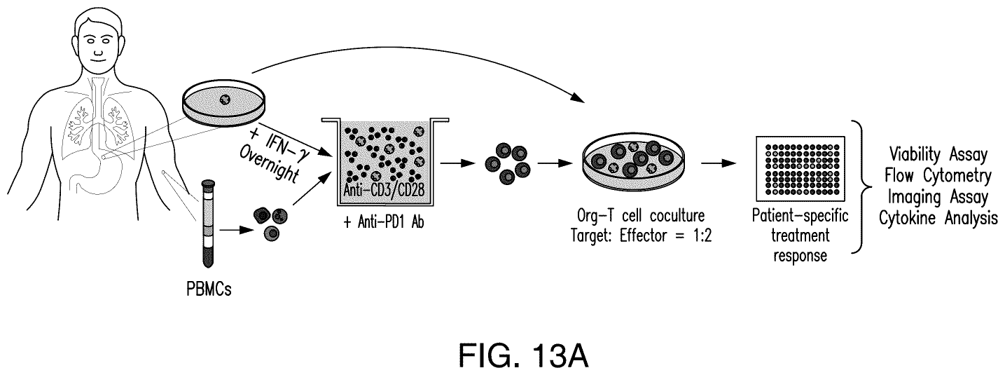

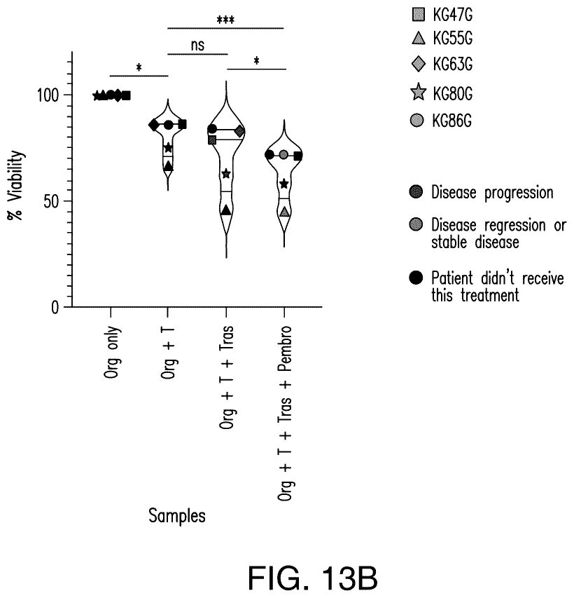

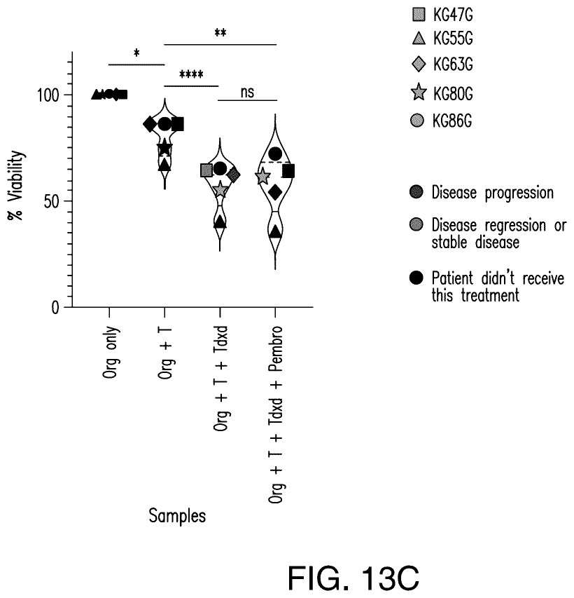

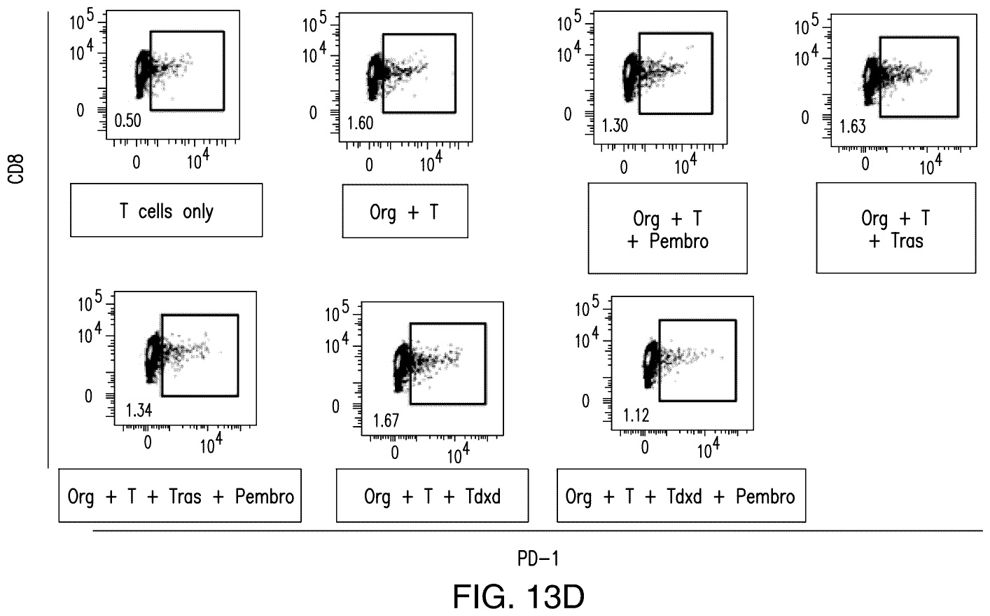

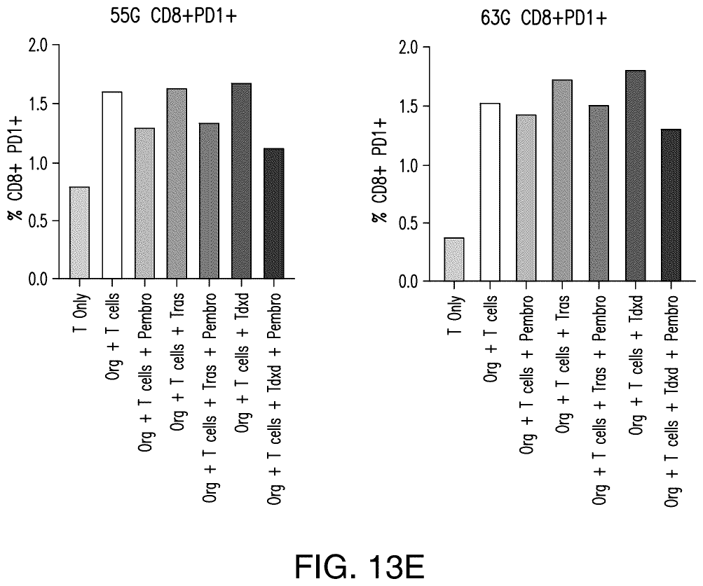

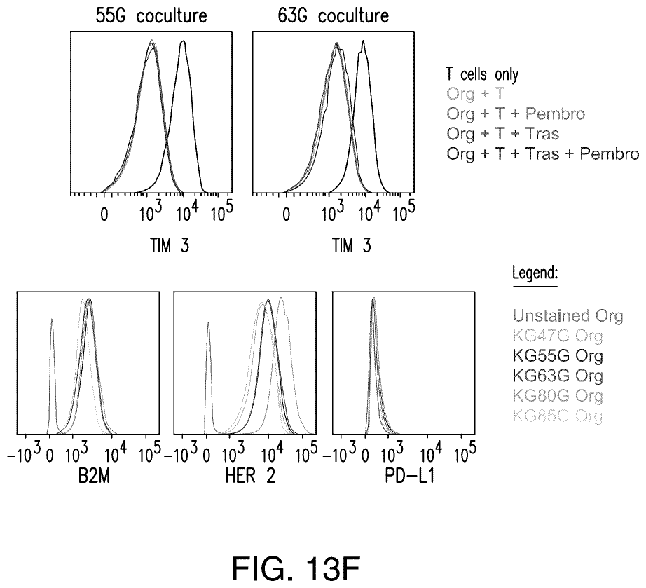

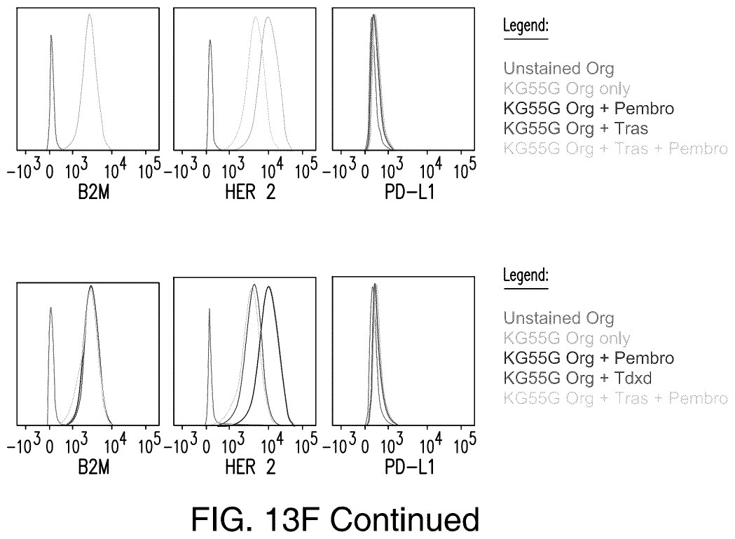

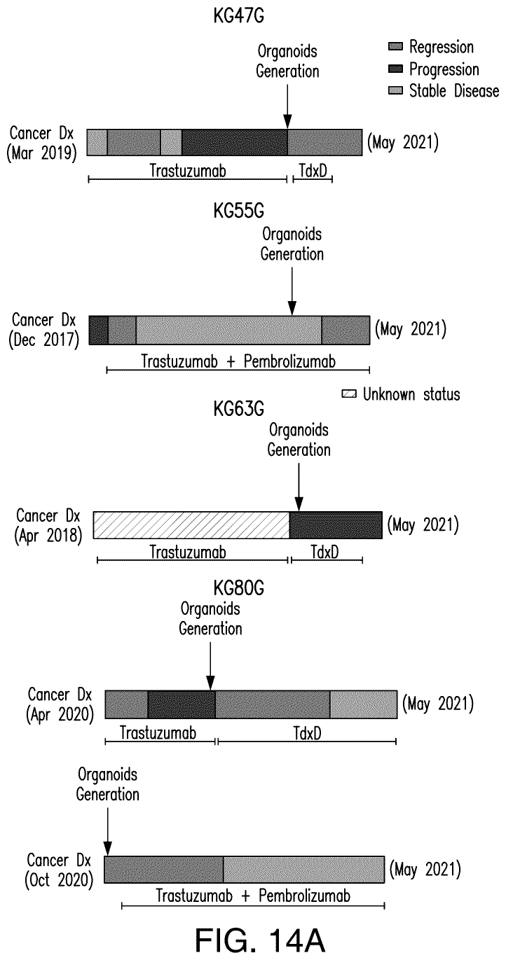

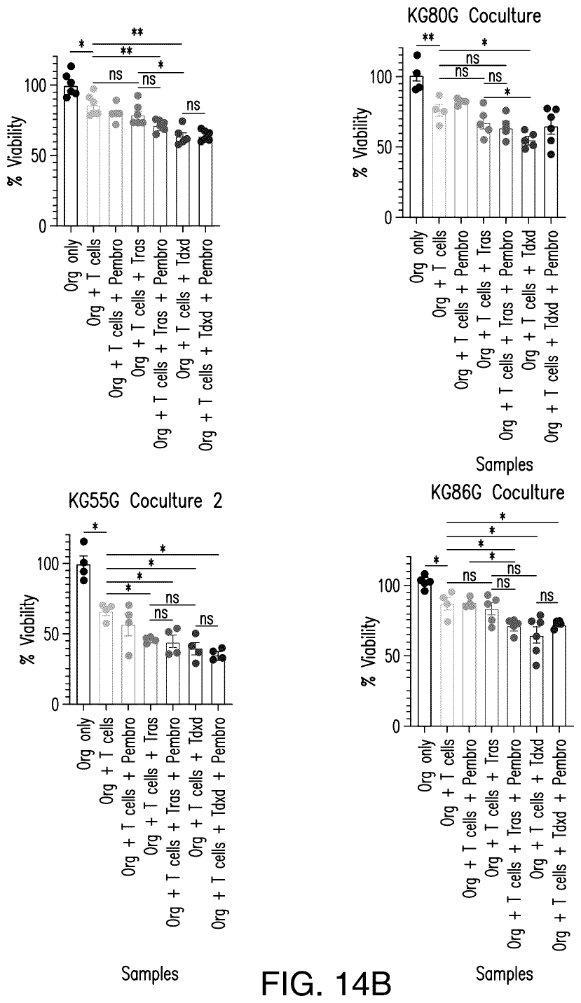

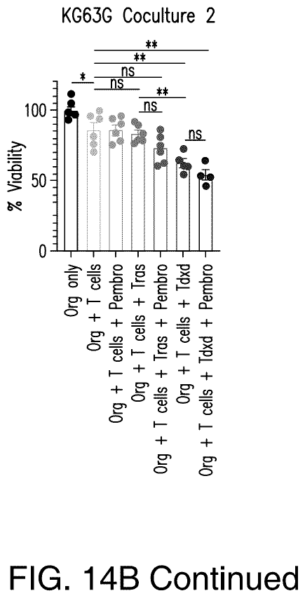

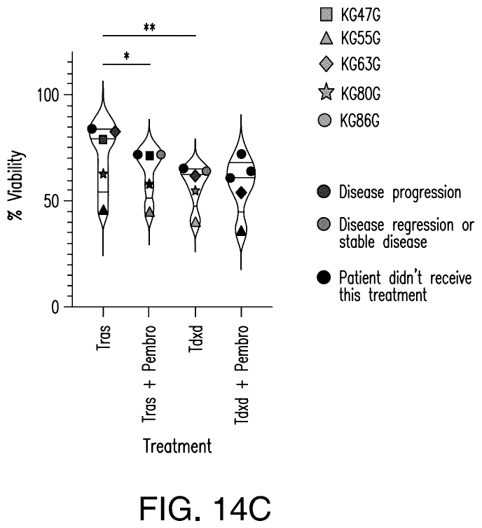

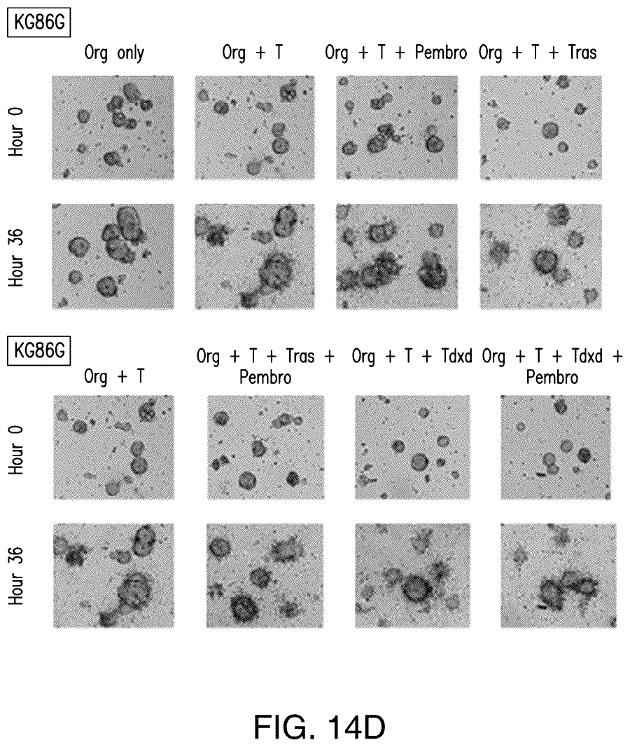

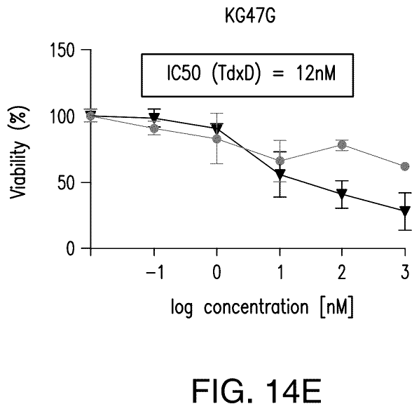

Additional experiments were performed studying the analysis of the combination treatment trastuzumab and pembrolizumab compared to trastuzumab alone according to the protocol of FIG. 13A. Patient specific organoids were generated after treatment with trastuzumab and were subsequently contacted with PBMCs followed by treatment with pembrolizumab (FIG. 14A). As shown in FIGS. 13B, 14B and 14D, trastuzumab in combination with pembrolizumab is more effective than trastuzumab alone in the organoid-T cell co-culture. In addition, pembrolizumab did not aid the cytotoxicity of trastuzumab-deruxtecan (TDxD) in the organoid-T cell co-culture (FIGS. 13C, 14C, 14D and 14E). As shown in FIGS. 13D and 13E, PD-1 expression increased in the organoid-T cell co-culture but was reduced upon the addition of pembrolizumab. In addition, TIM3 expression reduced in organoid-T cell co-culture (FIG. 13F). FIG. 13F also provides the expression of B2M, HER2 and PD-L1 in the organoid-T cell co-culture and under treatment conditions.

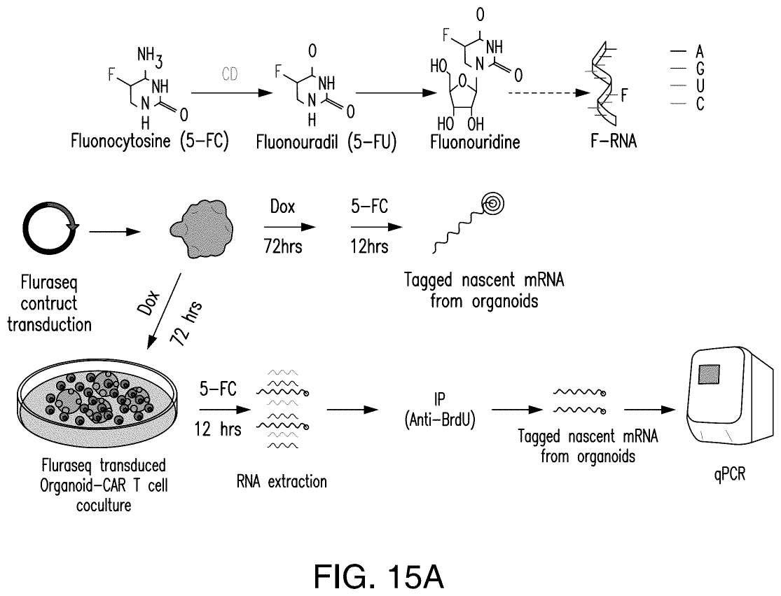

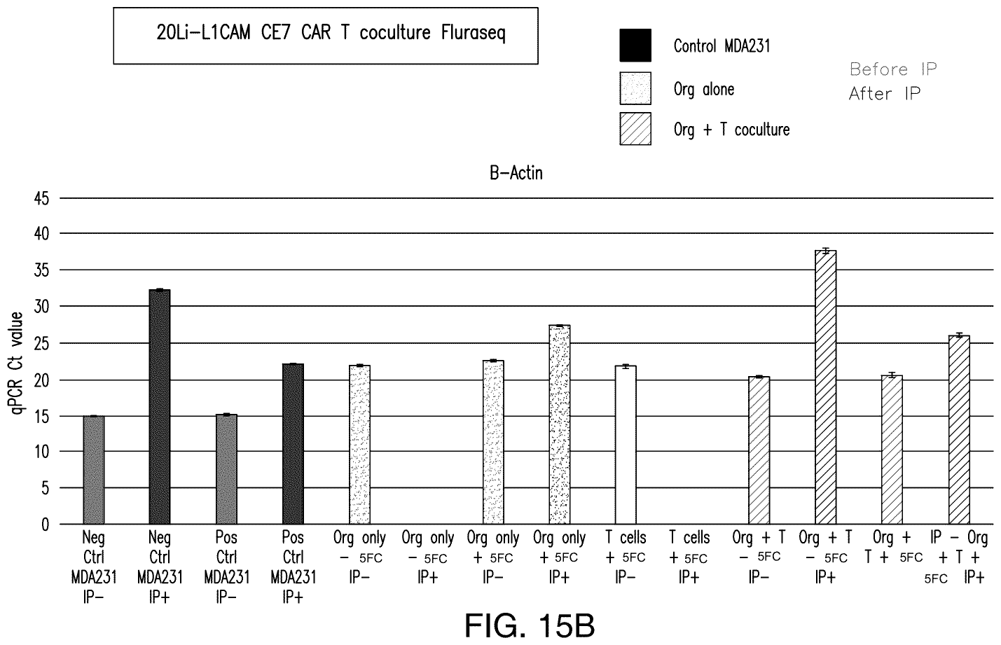

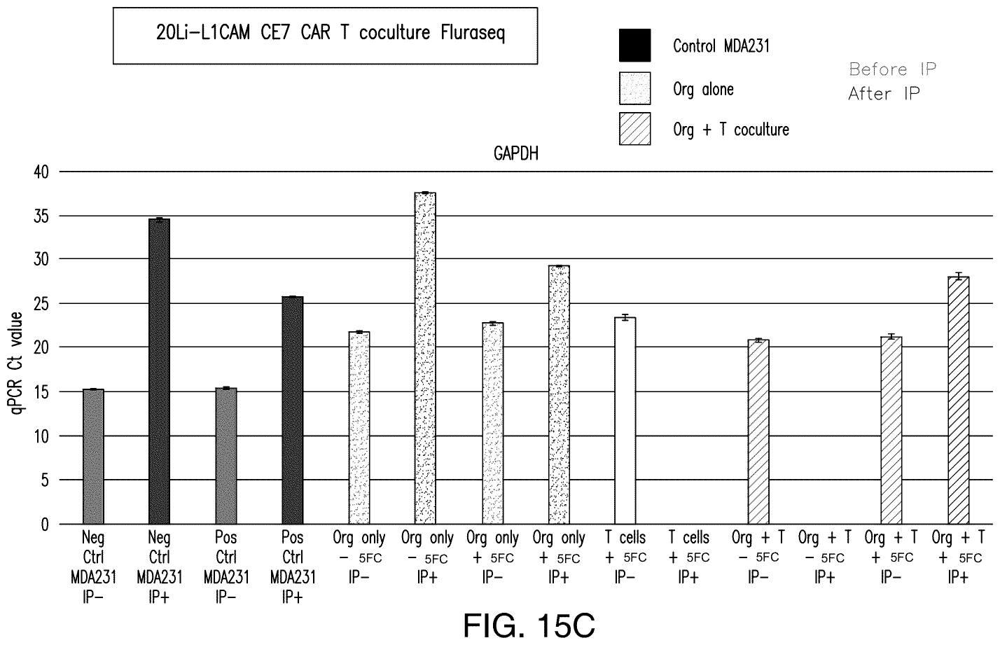

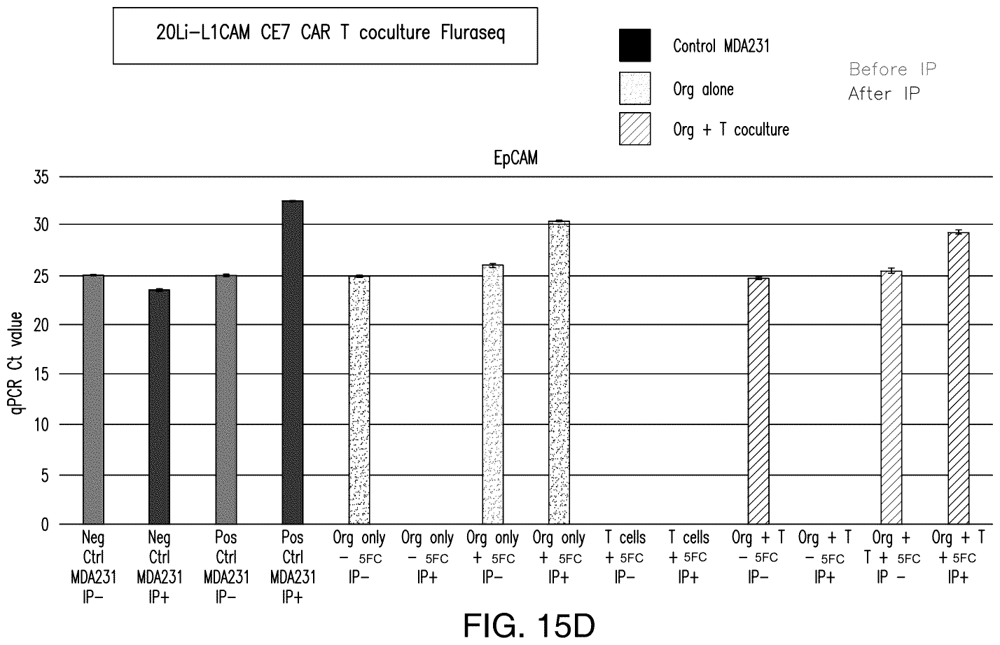

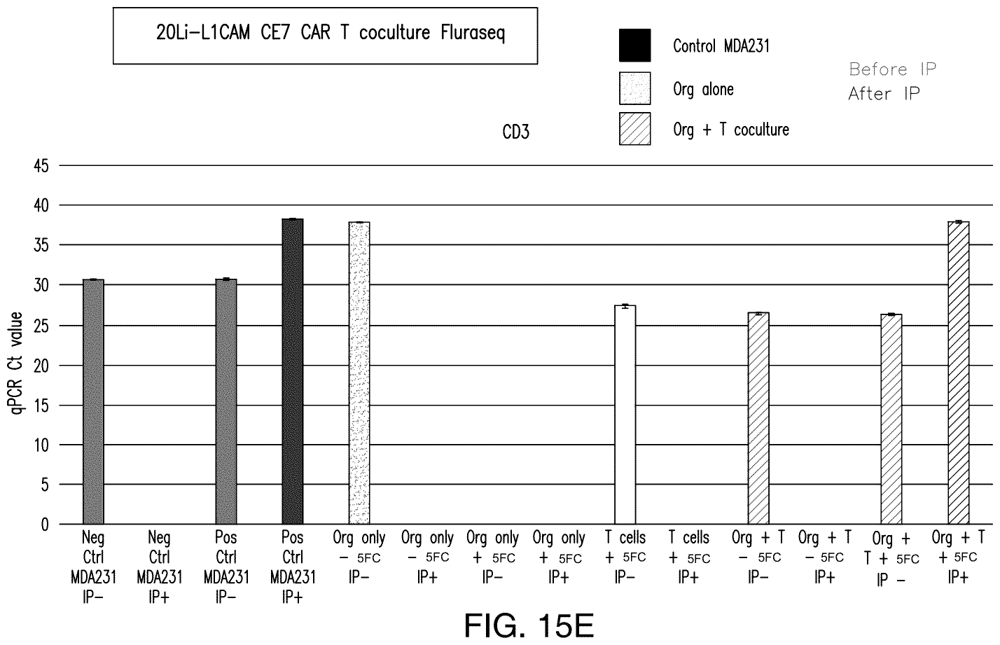

FIG. 15A provides the protocol for determining RNA expression levels of genes of interest. FIGS. 15B-15E shows the qPCR results for B-actin, GADPH, EpCAM and CD3 expression in the organoid-T cell co-cultures, respectively. These data show that the organoid-T cell co-cultures can be used to test treatment protocols and that gene expression can be determined in the organoid-T cell co-cultures.

### Example 2: Formation of Organoids with Bacterial Cells

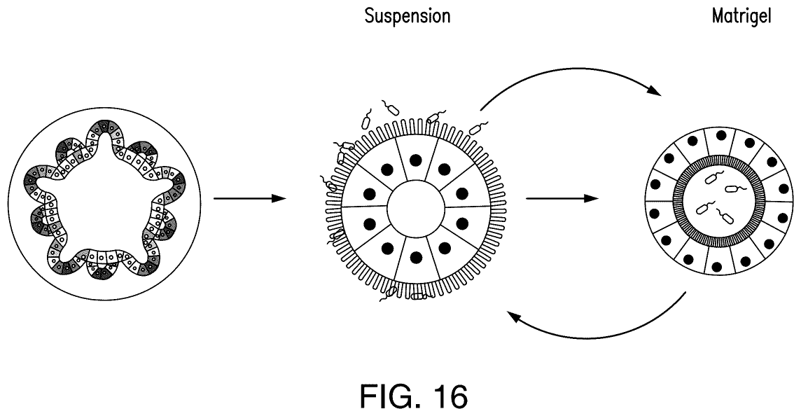

This Example discloses the generation of an organoid-bacterial cell co-culture. In particular, this Example discloses the generation of organoids that comprise bacterial cells in the luminal space of the organoids as shown in FIG. 16.

To generate organoid-bacterial cell co-cultures, organoids were grown from single cells for 7-14 days, then washed with ADF wash buffer containing no antibiotics, treated with TryPle for 5 minutes until small clumps were obtained. An aliquot was fully dissociated into single cells and counted. Organoids were seeded at 100,000 cells/well of a 6-well plate in suspension culture in organoid growth media (HISC medium (Advanced DMEM/F12 containing 100 ng/ml Wnt-3a (R&D; or conditioned medium from L-Wnt3A cells (ATCC)), 1 μg/ml R-Spondin1 (Peprotech; or conditioned medium from m-RSpo-Fc cells), 50 ng/ml EGF, 50 ng/ml Noggin (Peprotech), 10 nM gastrin (Sigma), 10 mM nicotinamide (Sigma), 500 nM A83-01 (Sigma), 10 μM SB202190, 10 mM HEPES, 2 mM glutamine, 2 mM N-acetylcysteine, 1 μM PGE2 (Sigma), 1:100 N2 (Invitrogen), 1:50 B27 (without vitamin A) and Y27632 (10 μM; Sigma)). Bacteria were grown overnight, then resceded and grown to log phase, counted and added to the organoid suspensions at an MOI of 100-400. To prevent bacterial overgrowth, gentamicin was added at a concentration of 0.005 μg/ml to 5 μg/ml. The organoid:bacteria suspension culture was incubated at 37° C. in a 5% CO₂ incubator. After 24-72 hours, typically 48 hours, in suspension culture, the organoid cell suspension was harvested from the 6 well plate, centrifuged at 100 g for 5′ at 4° C., washed 1× with ADF wash buffer and centrifuged again at 100 g for 5′ at 4° C. Organoids were plated in 50% basement membrane extract (corresponding to 200,000 cells at the start of the experiment) in 400 μL 50% basement membrane extract, divided into 10 drops of 40 μL each/well of a 6-well plate. 2 ml HISC media containing 100 μg/ml Primocin (InvivoGen) was added per well and the well was incubated at 37° C. in a 5% CO₂ for 5 days. After 5 days, organoids can once again be disrupted as above for reinoculation with a fresh batch of bacterial culture. This cycle can be repeated up to 6 months as needed for experimental purposes.

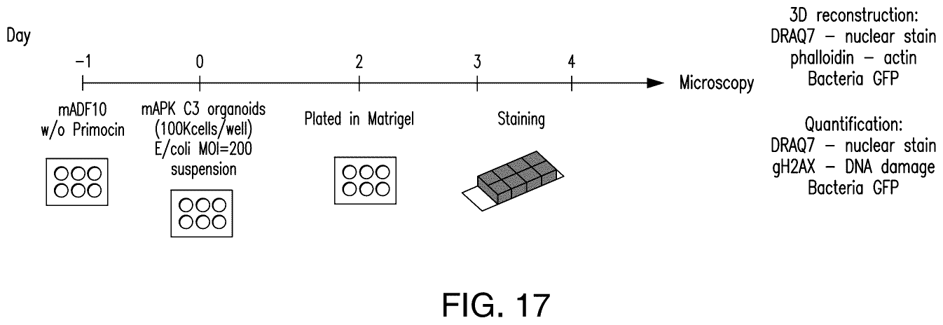

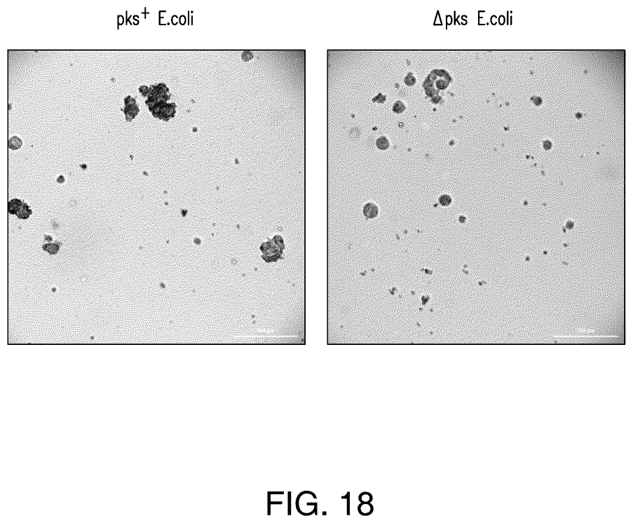

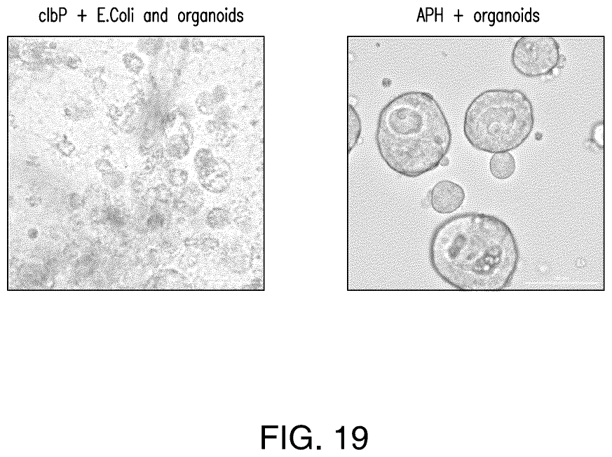

As shown in FIG. 17, the organoid-bacterial cell co-culture were generated by culturing organoids (about 100,000 cells/well) in the presence of E. coli cells (MOI=200). After 2 days of co-culture, the organoid-bacterial cells were plated in Matrigel followed by staining at day 3 or 4. The organoids were cultured in the presence of pks+ and Δpks E. coli as shown in FIG. 18. The organoids were also cultured in the presence of clbP+E. coli or Aphidicolin (APH) as shown in FIG. 19. Organoids and bacteria underwent cell death after plating in Matrigel.

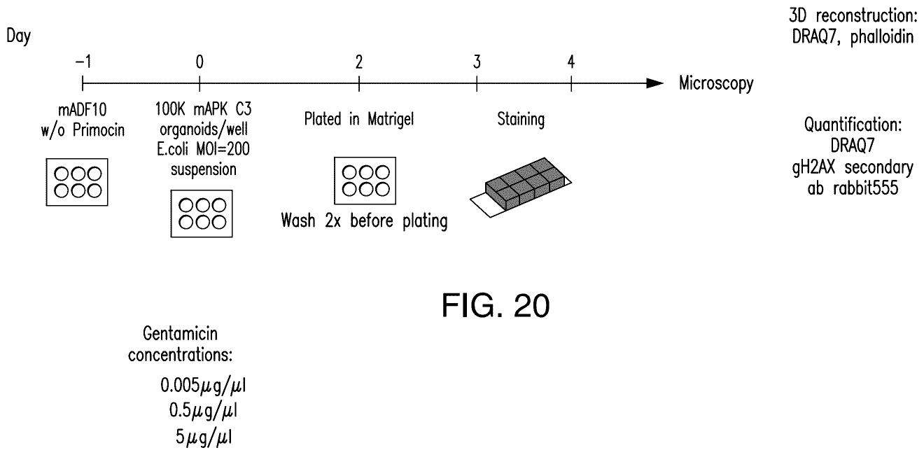

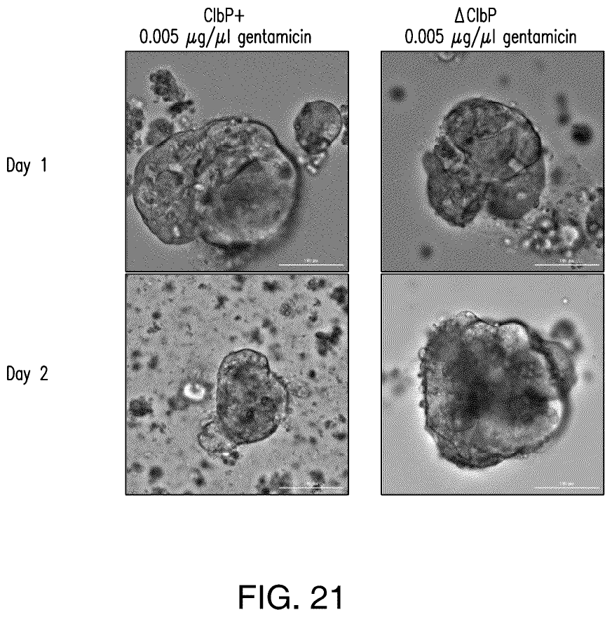

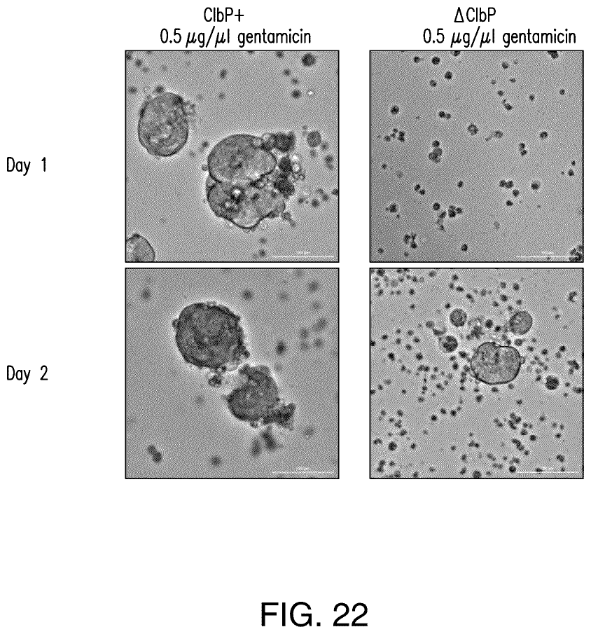

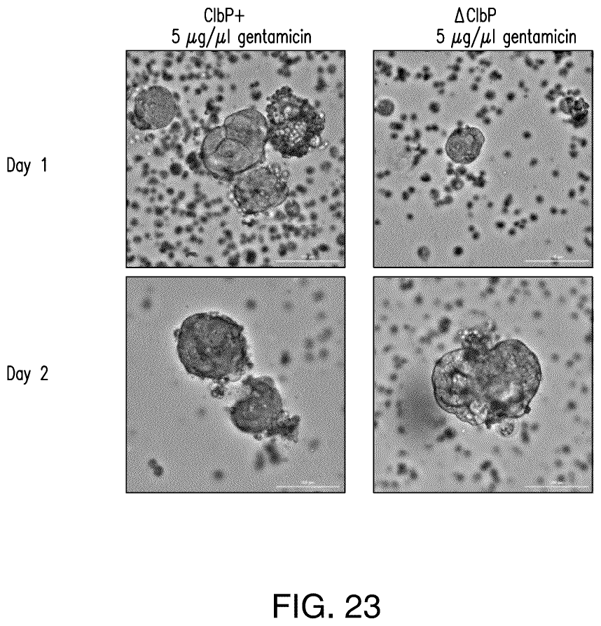

To prevent overgrowth of the co-cultures, either gentamycin was added at different concentrations and/or the co-cultures were washed two times before plating in Matrigel as shown in FIG. 20. Co-culture of organoids and ClbP+ or ΔClbP. E. coli were treated with 0.005 μg/μl (FIG. 21), 0.5 μg/μl (FIG. 22) or 5.0 μg/μl (FIG. 23) of gentamycin. The organoid-bacteria co-cultures appeared healthier at lower concentrations of gentamycin and in the absence of gentamycin. In addition, washing the co-cultures prior to plating in Matrigel was sufficient to prevent overgrowth. As shown in FIG. 24, the bacterial cells (ΔClbP+ E. coli), labeled in green, were observed inside the organoids treated with 0.005 μg/μl. These data show that organoids can be generated that have bacterial cells inside the lumen of organoids.

Although the presently disclosed subject matter and its advantages have been described in detail, it should be understood that various changes, substitutions and alterations can be made herein without departing from the spirit and scope of the present disclosure. Moreover, the scope of the present application is not intended to be limited to the particular embodiments of the process, machine, manufacture, and composition of matter, means, methods and steps described in the specification. As one of ordinary skill in the art will readily appreciate from the invention of the presently disclosed subject matter, processes, machines, manufacture, compositions of matter, means, methods, or steps, presently existing or later to be developed that perform substantially the same function or achieve substantially the same result as the corresponding embodiments described herein can be utilized according to the presently disclosed subject matter. Accordingly, the appended claims are intended to include within their scope such processes, machines, manufacture, compositions of matter, means, methods, or steps.

Various patents, patent applications, publications, product descriptions, protocols, and sequence accession numbers are cited throughout this application, the contents of which are incorporated herein by reference in their entireties for all purposes.

What is claimed is:

1. A method for testing a therapeutic agent comprising:
(a) contacting an organoid co-culture with a candidate therapeutic agent;
(b) detecting the presence of a change in the organoid co-culture that is indicative of the effectiveness of the therapeutic agent; and
(c) identifying the candidate therapeutic agent as likely to have a therapeutic effect if the change is detected.

2. The method of claim 1, wherein the organoid co-culture comprises an immune cell or a bacterial cell.

3. The method of claim 2, wherein the organoid co-culture comprises a bacterial cell.

4. The method of claim 2, wherein the organoid co-culture comprises an immune cell.

5. The method of claim 4, wherein the immune cell is a T cell.

6. The method of claim 5, wherein the T cell is a CAR T cell.

7. The method of any one of claims 4-6, wherein the ratio of the total number cells of the organoid to the total number of immune cells is from about 1:1 to about 20:1.

8. The method of any one of claims 1-7, wherein the change is a change in cell viability, cell proliferation, organoid size, morphology, mutational status, epigenetic state, RNA expression levels and/or protein expression.

9. The method of claim 8, wherein the change is a change in cell viability.

10. The method of claim 9, wherein the change in cell viability is a change in the viability of the cells of the organoid.

11. The method of any one of claims 1-10, wherein the organoid is generated from cancer cells of a subject.

12. The method of claim 11, wherein the immune cells are derived from the subject.

13. The method of any one of claims 1-12, wherein the organoid comprises one or more colorectal or esophagogastric cancer cells.

14. The method of any one of claims 1-13, wherein the organoid comprises one or cancer cells that express high levels of LICAM.

15. The method of claim 6, wherein the CAR T cell is a LICAM CAR T cell.

16. A method for testing the efficacy of a candidate modified immune cell comprising:
(a) contacting an organoid with the candidate modified immune cell to generate an organoid-immune cell co-culture;
(b) detecting the presence of a change in the organoid-immune cell co-culture that is indicative of the effectiveness of the candidate modified immune cell; and
(c) identifying the candidate modified immune cell as likely to have a therapeutic effect if the change is detected.

17. The method of claim 16, wherein the modified immune cell is an immune cell genetically engineered to express an antigen-binding receptor.

18. The method of claim 17, wherein antigen-binding receptor is a chimeric antigen receptor (CAR).

19. The method of any one of claims 16-18, wherein the change is a change in cell viability, cell proliferation, organoid size, morphology, mutational status, epigenetic state, RNA expression levels and/or protein expression.

20. The method of claim 19, wherein the change is a change in cell viability.

21. The method of any one of claims 16-20, wherein the organoid is generated from cancer cells of a subject.

22. The method of claim 21, wherein modified immune cells are derived from the subject.

23. The method of any one of claims 16-22, wherein the organoid comprises one or more colorectal or esophagogastric cancer cells.

24. The method of any one of claims 16-23, wherein the organoid comprises one or cancer cells that express high levels of LICAM.

25. The method of any one of claims 16-24, wherein the modified immune cell is a CAR T cell.

26. The method of claim 25, wherein the CAR T cell is a LICAM CAR T cell.

27. A method for analyzing a bacterial species comprising:
(a) culturing an organoid with a candidate bacterial species to generate an organoid-bacterial cell co-culture;
(b) detecting the presence of a change in the organoid-bacterial cell co-culture that is indicative of the oncogenic potential of the bacterial species; and
(c) identifying the bacterial species as likely to have an oncogenic effect if the change is detected.

28. The method of claim 27, wherein the organoid co-culture further comprises an immune cell.

29. The method of claim 28, wherein the immune cell is a T cell.

30. The method of any one of claims 27-29, wherein the change is a change in cell viability, cell proliferation, organoid size, morphology, mutational status, epigenetic state, RNA expression levels and/or protein expression.

31. The method of claim 30, wherein the change is a change in mutational status.

32. The method of claim 31, wherein the change in mutational status is a change in mutational status of one or more cells of the organoid.

33. The method of any one of claims 27-32, wherein the organoid is generated from normal cells of a subject.

34. A method for analyzing a bacterial species comprising:
(a) providing an organoid-bacterial cell co-culture comprising cancer cells and one or more candidate bacteria;
(b) contacting the organoid-bacterial cell co-culture with a therapeutic agent;
(c) detecting the presence of a change in the organoid-bacterial cell co-culture that is indicative of the potential of the bacterial species to increase the effectiveness of the therapeutic agent; and
(d) identifying the bacterial species as likely to increase the effectiveness of the therapeutic agent if the change is detected.

35. The method of claim 34, wherein the organoid-bacterial cell co-culture further comprises an immune cell.

36. The method of claim 34, wherein the therapeutic agent is an immune cell.

37. The method of claim 35 or 36, wherein the immune cell is a T cell.

38. The method of any one of claims 34-37, wherein the change is a change in cell viability, cell proliferation, organoid size, morphology, mutational status, epigenetic state, RNA expression levels and/or protein expression.

39. The method of claim 38, wherein the change is a change is a change in cell viability.

40. The method of claim 39, wherein the change is a change in cell viability is a decrease in cell viability of the cells of the organoid.

41. The method of any one of claims 34-40, wherein the organoid is generated from cancer cells of a subject.

42. The method of any one of claims 35-41, wherein the immune cell is a modified immune cell.

43. The method of claim 42, wherein the modified immune cell is a CAR T cell.

44. The method of any one of claims 1-43, wherein the organoid co-culture, organoid-immune cell co-culture and/or the organoid-bacterial cell co-culture is cultured in a media comprising:
(a) from about 1 to about 500 ng/ml of Wnt3a;
(b) from about 1 to about 500 ng/ml of Noggin;
(c) from about 1 to about 500 ng/ml of EGF;
(d) from about 0.01 to about 1,000 ng/ml of R-spondin-1; or
(e) a combination of any one of (a)-(d).

45. The method of claim 44, wherein the organoid co-culture is cultured in a media comprising about 100 ng/ml Wnt-3a, about 1,000 ng/ml R-spondin-1, about 50 ng/ml Noggin and about 50 ng/ml EGF.

46. The method of any one of claims 1-45, wherein the organoid co-culture, organoid-immune cell co-culture and/or the organoid-bacterial cell co-culture is cultured in a media comprising an antibiotic at a concentration from about 0.005 μg/μl to about 5 μg/μl.

47. The method of claim 46, wherein the concentration of the antibiotic is from about 0.001 μg/μl to about 1.0 μg/μl.

48. An organoid co-culture comprising one or more organoid cells and one or more immune cells or bacterial cells, wherein the ratio of organoid cells to immune or bacterial cells is from about 1:1 to about 20:1.

49. A kit for performing the method of any one of claims 1-47.

50. A kit comprising the organoid co-culture of claim 48.
